(19) United States
(12) Patent Application Publication
(10) Pub. No.: US 2016/0207860 A1
(43) Pub. Date: Jul. 21, 2016

(54) PROCESS FOR PREPARING SYNTHETIC PARA-EUGENOL

(71) Applicant: SABIC GLOBAL TECHNOLOGIES B.V., Bergen op Zoom (NL)

(72) Inventors: Shubashree Swaminathan, Bangalore, Karnataka (IN); Ganapathy Bhotla Venkata Ramanarayanan, Bangalore, Karnataka (IN)

(73) Assignee: Sabic Global Technologies B.V., Bergen op Zoom (NL)

(21) Appl. No.: 14/908,135

(22) PCT Filed: Jul. 30, 2014
(86) PCT No.: PCT/IB2014/063563

§ 371 Date: Jan. 28, 2016

Related U.S. Application Data
(60) Provisional application No. 61/859,847, filed on Jul. 30, 2013.

(51) Int. Cl. C07C 41/18; C07C 41/30; C07C 51/09

(52) U.S. Cl. CPC C07C 41/18; C07C 51/09; C07C 41/30

(57) ABSTRACT

Processes are provided for preparing synthetic para-eugenol and polysiloxane-polycarbonate copolymers including the synthetic para-eugenol. In an embodiment, a process for synthesizing para-eugenol can comprise: a) hydrolyzing methyl 5-allyl-3-methoxysalicylate to form 5-allyl-3-methoxysalicylic acid; b) decarboxylating the 5-allyl-3-methoxysalicylic acid to form a product comprising para-eugenol. The polysiloxane-polycarbonate copolymer prepared by the process may be isolated by, for example, anti-solvent precipitation followed by vacuum drying.

### TECHNICAL FIELD

The present disclosure is directed to processes for preparing synthetic para-eugenol and polysiloxane-polycarbonate copolymers comprising the synthetic para-eugenol.

### BACKGROUND

Eugenol end-capped polysiloxanes are often used as the source of the polysiloxane blocks, as they include terminal phenolic groups to copolymerize with other compounds to form the polycarbonate blocks. Para-eugenol is present in varying amounts in a number of essential oils such as clove, cinnamon, pimento, calamus, camphor, sassafras and nutmeg, and has also been found in other plant species. However, commercial para-eugenol is prepared almost exclusively by alkali extraction from clove leaf and bud oils. As clove is a seasonal crop, the supply, availability and cost of para-eugenol can fluctuate significantly.

### SUMMARY

Disclosed are processes for synthesizing para-eugenol, and polysiloxane-polycarbonate copolymers comprising the synthetic para-eugenol. Other aspects and embodiments will become apparent in light of the following description.

### DETAILED DESCRIPTION

The present disclosure is directed to processes for preparing synthetic para-eugenol. This compound has a wide variety of uses, for example in the preparation of eugenol end-capped polysiloxanes. To avoid uncertainty, fluctuating availability and cost associated with using naturally-derived eugenol as an end-capping group, the inventors have identified synthetic routes to para-eugenol. While certain synthetic methods for preparing para-eugenol are known, they are not attractive for industrial applications due to operational difficulty on large scales, as well as cost. The present inventors have developed processes that are suitable for production on a large scale, and at a cost that is competitive with that for production of para-eugenol from natural sources.

The disclosure provides several methods for preparing synthetic para-eugenol. One process involves initial hydrolysis of methyl 5-allyl-3-methoxysalicylate to form 5-allyl-3-methoxysalicylic acid, and subsequent decarboxylation to form para-eugenol. A second process involves direct decarboxylation of methyl 5-allyl-3-methoxysalicylate. A third process involves direct C-allylation of guaiacol.

The present disclosure is also directed to processes for preparing polysiloxane-polycarbonate copolymers comprising the synthetic para-eugenol.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

“About” is intended to include the degree of error associated with measurement of the particular quantity based upon the equipment available at the time of filing the application.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Alkali metal” as used herein refers to lithium, sodium, potassium, rubidium or cesium. “Alkaline earth metal” as used herein refers to beryllium, magnesium, calcium, strontium and barium. “Alkyl” as used herein may mean a linear, branched, or cyclic hydrocarbyl group, such as a methyl group, ethyl group, n-propyl group, isopropyl group, n-butyl group, isobutyl group, tert-butyl group, n-pentyl group, isopentyl group, n-hexyl group, isohexyl group, cyclopentyl group, cyclohexyl group, or the like. “Amide” as used herein refers to a compound having a group —C(O)N(R)₂, wherein each R can independently be H or non-H, such as alkyl, aryl, and the like. “Amine” as used herein refers to primary, secondary, and tertiary amines having, e.g., the formula N(R)₃ wherein each R can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein, as well as diamines an polyamines that include two or more —N(R)₂ groups. “Aryl” as used herein may mean substituted or unsubstituted aryl radicals containing from 6 to 36 ring carbon atoms. Examples of aryl include, but are not limited to, a phenyl group, a naphthyl group, a bicyclic hydrocarbon fused ring system, or a tricyclic hydrocarbon fused ring system wherein one or more of the rings are a phenyl group. “Catalytic amount” as used herein refers to a sub-stoichiometric amount of a catalyst compound compared to one or more reactants, which is effective to increase the rate of reaction relative to the same reaction without the catalyst. “D45 polydimethylsiloxane”, “D45 siloxane” or “D45 block” as used herein refers to the polydiorganosiloxane blocks which comprise the siloxane-containing repeating units of a polysiloxane monomer, oligomer or polymer in which there is an average of 45 siloxane repeating units. “Halide” as used herein may mean a fluoride, chloride, bromide or iodide anion. “Mineral acid” as used herein refers to an acid derived from one or more inorganic compounds. Non-limiting examples of inorganic acids include hydrochloric acid, hydrobromic acid, nitric acid, sulfuric acid, phosphoric acid, boric acid and perchloric acid.

Unless otherwise indicated, each of the foregoing groups may be unsubstituted or substituted, provided that the substitution does not significantly adversely affect synthesis, stability, or use of the compound.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

One process for preparing para-eugenol involves hydrolysis of methyl 5-allyl-3-methoxysalicyate to form 5-allyl-3-methoxysalicylic acid, followed by decarboxylation to produce a product comprising para-eugenol, as shown in Scheme 1. This process has almost 100% selectivity for producing the para-isomer of eugenol. The process therefore comprises: a) hydrolyzing methyl 5-allyl-3-methoxysalicylate to form 5-allyl-3-methoxysalicylic acid; b) decarboxylating the 5-allyl-3-methoxysalicylic acid to form a product comprising para-eugenol.

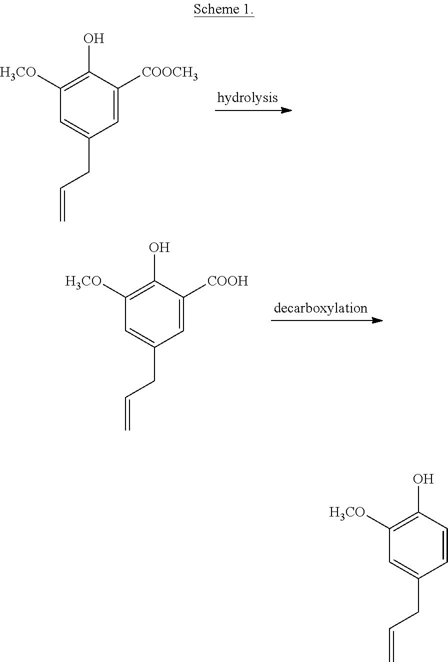 [SMILES: C=CCc1cc(OC)c(O)c(C(=O)OC)c1 ; C=CCc1cc(OC)c(O)c(C(=O)O)c1 ; C=CCc1ccc(O)c(OC)c1]

Hydrolysis: Step a) of the process comprises hydrolyzing methyl 5-allyl-3-methoxysalicylate to form 5-allyl-3-methoxysalicylic acid. The methyl 5-allyl-3-methoxysalicylate is commercially available from a wide variety of suppliers, or may be prepared by standard methods known in the art.

Step a) may comprise refluxing a mixture of methyl 5-allyl-3-methoxysalicylate in an aqueous base. The aqueous base may comprise any suitable base, such as an alkali or alkaline earth metal hydroxide such as sodium hydroxide or potassium hydroxide. The aqueous base may comprise any suitable amount of the base. For example, the aqueous base may be a solution of 1 wt % to 25 wt %, or 5 wt % to 20 wt %, or 10 wt % to 15 wt %, e.g., about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % of the base in water. In a suitable embodiment, the aqueous base may comprise a solution of about 10 wt % sodium hydroxide in water.

During refluxing, the reaction may be monitored by any suitable means to follow the progress of the hydrolysis, e.g. by thin-layer chromatography (TLC) or by high performance liquid chromatography (HPLC). The mixture may be refluxed for any suitable period of time to effect complete or near-complete hydrolysis. For example, the mixture may be refluxed for 1 hour to 5 hours, e.g., about: 1 hour, 1.5 hours, 2 hours, 2.5 hours, 3 hours, 3.5 hours, 4 hours, 4.5 hours or 5 hours as needed. In embodiments, the mixture may be refluxed for about 2-3 hours.

Following refluxing, the mixture may be cooled, e.g., to room temperature. The cooling may be effected by simply removing a heating source and allowing the reaction to equilibrate to ambient temperature, or by providing a source of cooling such as an ice bath. The mixture may then be neutralized or acidified with an acid. For example, the mixture may be neutralized or acidified with an acid such as a mineral acid. An exemplary mineral acid is hydrochloric acid. The acid may be of any suitable concentration. For example, the acid may be a solution of 1 wt % to 25 wt %, or 5 wt % to 20 wt %, or 10 wt % to 15 wt %, e.g., 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % of the acid in water. The mixture may be neutralized or acidified by either adding the mixture to a solution of the acid, or by adding the acid to the mixture. The neutralization/acidification results in precipitation of the product 5-allyl-3-methoxysalicylic acid, which can be isolated by filtration. The filtered product may be washed, e.g., with water, and dried to produce the final product. For example, the product may be dried using heat and/or vacuum.

Step a) may produce 5-allyl-3-methoxysalicylic acid in a yield of at least about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In embodiments, step a) may produce 5-allyl-3-methoxysalicylic acid in a yield of at least about 95%, at least about 96%, at least about 97%, at least about 98% or at least about 99%.

Decarboxylation: Step b) of the process comprises decarboxylating the 5-allyl-3-methoxysalicylic acid to form para-eugenol. In a suitable embodiment, the 5-allyl-3-methoxysalicylic acid is produced in step a). However, the 5-allyl-3-methoxysalicylic acid may also be obtained commercially and used directly in a process comprising step b).

Step b) may comprise heating a mixture of 5-allyl-3-methoxysalicylic acid in a polar aprotic solvent, a base, or a combination thereof. Exemplary polar aprotic solvents include but are not limited to N,N-dimethylformamide (DMF), dimethylsulfoxide (DMSO), and the like. The base may be, e.g., a substituted or unsubstituted aromatic amine, including primary, secondary and tertiary amines. For example, one suitable aromatic amine is N,N-dimethylaniline (DMA). In embodiments such embodiments, the ratio by weight of the 5-allyl-3-methoxysalicylic acid to the N,N-dimethylaniline may be from about 1:0.1 to about 1:3, e.g., about 1:0.1 to 1:2. In some embodiments the ratio by weight of the 5-allyl-3-methoxysalicylic acid to the N,N-dimethylaniline is about 1:0 (in other words, no N,N-dimethylaniline is included), which may result in a lower purity of the isolated crude para-eugenol.

In step b), the mixture may further comprise a metal salt, such as an alkali metal halide. Alkali metal halides include, for example, lithium chloride, lithium bromide, sodium chloride, sodium bromide, potassium chloride and potassium bromide. In a suitable embodiment, the metal salt may comprise lithium chloride. In such embodiments, the ratio by weight of the 5-allyl-3-methoxysalicylic acid to the lithium chloride may be about 1:0.1 to about 1:1, or about 1:0.1 to about 1:0.5. In some embodiments, the ratio by weight of the 5-allyl-3-methoxysalicylic acid to the lithium chloride may be about 1:0 (in other words, no lithium chloride is included), where the formation of an ester impurity is greater than (>)1%, which may eventually impact the final isolated yield of para-eugenol.

In some embodiments of step b), the mixture may be heated to reflux. As a skilled artisan will appreciate, the temperature of the reflux will depend on the solvent and/or base selected. In some embodiments, the mixture may be heated until all of the components melt and the mixture becomes a homogenous liquid. The mixture may be heated for about 1 hour to about 12 hours. For example, the mixture may be heated for 1 to 12 hours, e.g., about: 1, 2, 3, 4, 5, 6, 7, 8, 9, 10, 11 or 12 hours. In embodiments, the mixture may be heated for about 3-8 hours or 5-6 hours. During heating, the reaction may be monitored by any suitable means to follow the progress of the decarboxylation, e.g. by TLC or HPLC. The mixture may be refluxed for any suitable period of time to effect complete or near-complete decarboxylation.

Following the heating step, step b) may further comprise cooling the mixture (e.g., to room temperature) and diluting the mixture with an organic solvent. The cooling may be effected by simply removing a heating source and allowing the reaction to equilibrate to ambient temperature, or by providing a source of cooling such as an ice bath. The solvent may be a polar or non-polar solvent, such as diethyl ether, chloroform, methylene chloride, ethyl acetate, benzene, toluene, pentane, hexane, cyclohexane, 1,2-dichloroethane, or any mixture thereof. In an embodiment, the organic solvent may be 1,2-dichloroethane. To the diluted organic mixture may be added a solution of a mineral acid, such as aqueous hydrochloric acid. The acid may be added such that the temperature is maintained at less than about 25 C (e.g., about room temperature). For example, the acid may be added dropwise. The resulting mixture may be stirred, followed by separation of the organic layer. The organic layer may be passed through an ion exchange resin (IER) to remove any residual base, if present. For example, a suitable resin is an acidic resin, such as an acidic sulfonated polystyrene resin cross-linked with divinyl benzene.

The organic solvent may be removed from the final mixture, e.g., by rotary evaporation or distillation. Following solvent removal, the para-eugenol product may be purified by distillation, such as high-vacuum distillation, to provide the final product. Other methods of purification may also be used, such as column chromatography.

The process may provide para-eugenol in a yield of at least about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In embodiments, the process may produce para-eugenol in a yield of at least about 85%, at least about 86%, at least about 87%, at least about 88%, at least about 89%, or at least about 90%. The purity of the para-eugenol product may be at least about 98% or at least about 99%. For example, the purity of the para-eugenol product may be about: 99.0%, 99.1%, 99.2%, 99.3%, 99.4%, 99.5%, 99.6%, 99.7%, 99.8%, or 99.9%.

The process may further comprise synthesizing a eugenol end-capped polydiorganosiloxane, such as a eugenol end-capped polydimethylsiloxane, using the product comprising para-eugenol. Such a compound may be synthesized using any means known in the art, such as a hydrosilylation reaction. For example, a suitable method is described in U.S. Pat. No. 6,072,011. In a suitable reaction, a cyclotetrasiloxane (e.g., octamethylcyclotetrasiloxane) and a disiloxane (e.g., tetramethyldisiloxane) may be combined in the presence of an equilibration catalyst (e.g., an acid clay catalyst) and heated. Following this initial reaction, the product may be combined with the product comprising para-eugenol, and a platinum catalyst (e.g., Karstedt's Pt catalyst), to produce the eugenol end-capped polydiorganosiloxane.

Another process for preparing para-eugenol involves direct decarboxylation of methyl 5-allyl-3-methoxysalicyate, as shown in Scheme 2. This process has an advantage of being performed in a single step. The process therefore comprises: decarboxylating methyl 5-allyl-3-methoxysalicylic acid to form a product comprising para-eugenol.

In this process, all amounts described as equivalents are intended to refer to molar equivalents, with the number of moles of methyl 5-allyl-3-methoxysalicyate being one equivalent.

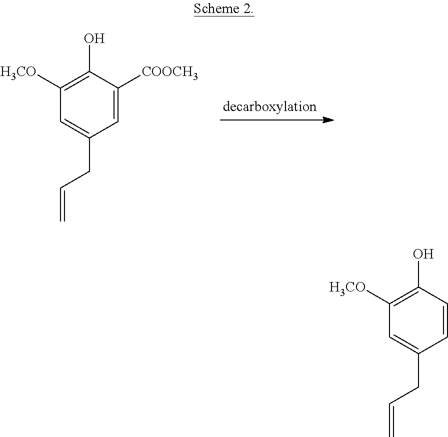 [SMILES: C=CCc1cc(OC)c(O)c(C(=O)OC)c1 ; C=CCc1ccc(O)c(OC)c1]

The process may comprise providing a mixture of methyl 5-allyl-3-methoxysalicylate and a catalyst, in a polar aprotic solvent or a base, and heating the mixture. The methyl 5-allyl-3-methoxysalicylate is commercially available from a wide variety of suppliers, or may be prepared by standard methods known in the art.

The catalyst may be at least one of alkali metal halides, aqueous mineral acids, and salts of aromatic amines. For example, the catalyst may be an alkali metal halide, such as lithium chloride or sodium chloride. A suitable catalyst may be lithium chloride. The catalyst may also be a mineral acid, such as hydrochloric acid. The catalyst may also be an aromatic amine salt. For example, the catalyst may be a salt of a primary, secondary or tertiary aniline, such as a hydrochloride of aniline or N,N-dimethylaniline.

The amount of the catalyst may be about 1.0 equivalent or less based on the amount of methyl 5-allyl-3-methoxysalicylic acid. For example, the catalyst may be included in the mixture at an amount of 0.50 to 1.00 equivalent, e.g., about: 1.00, 0.95, 0.90, 0.85, 0.80, 0.75, 0.70, 0.65, 0.60, 0.55, 0.50 equivalents. In embodiments, the molar ratio of the methyl 5-allyl-3-methoxysalicylate to the catalyst may be about 1:1.

Exemplary polar aprotic solvents include but are not limited to DMF and DMSO, and the like. The base may be, e.g., a substituted or unsubstituted aromatic amine, including primary, secondary and tertiary amines. For example, one suitable aromatic amine DMA.

The mixture of the methyl 5-allyl-3-methoxysalicylate, catalyst and polar aprotic solvent or a base may be heated to any suitable temperature to effect the decarboxylation. In embodiments, the mixture may be heated to reflux. As a skilled artisan will appreciate, the temperature of the reflux will depend on the solvent and/or base selected. The mixture may be heated (e.g., refluxed) for about 3 hours to about 12 hours. For example, the mixture may be heated for about 3, 4, 5, 6, 7, 8, 9, 10, 11 or 12 hours. During heating, the reaction may be monitored by any suitable means to follow the progress of the decarboxylation, e.g. TLC or HPLC. The mixture may be refluxed for any suitable period of time to effect complete or near-complete decarboxylation.

Following heating, the mixture may be cooled, e.g., to room temperature. The cooling may be effected by simply removing a heating source and allowing the reaction to equilibrate to ambient temperature, or by providing a source of cooling such as an ice bath. The mixture may then be quenched with water or with an aqueous acid. For example, the mixture may be neutralized with an acid such as a mineral acid. An exemplary mineral acid is hydrochloric acid. The acid may be of any suitable concentration. For example, they acid may be a solution of 1 wt % to 25 wt %, or 5 wt % to 20 wt %, or 10 wt % to 15 wt %, e.g., about: 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % of the acid in water. The mixture may be neutralized by either adding the mixture to a solution of the acid, or by adding the acid to the mixture. The quenched reaction mixture may then be extracted with an organic solvent to provide an organic extract. The organic solvent may be a polar or non-polar solvent. For example, the organic solvent may be diethyl ether, chloroform, methylene chloride, ethyl acetate, benzene, toluene, pentane, hexane, cyclohexane, 1,2-dichloroethane, or any mixture thereof. In some embodiments, the organic solvent may be diethyl ether. The organic extract may be dried to remove any residual water. For example, the organic extract may be dried over sodium sulfate or magnesium sulfate, and decanted or filtered. The solvent may then be removed to provide a crude product.

If desired, the crude product may be further purified. For example, the crude product can be purified using distillation (e.g., high vacuum distillation), column chromatography, or a combination thereof.

The process may produce para-eugenol as the major product. The process may provide para-eugenol in a yield of at least about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The purity of the para-eugenol product may be at least about 98% or at least about 99%.

The process may further comprise synthesizing a eugenol end-capped polydiorganosiloxane, such as a eugenol end-capped polydimethylsiloxane, using either the crude product or the purified product comprising para-eugenol. Such a compound may be synthesized using any means known in the art, such as a hydrosilylation reaction. Suitable methods are described above and herein.

Guaiacol is easily obtained synthetically via well-known methods or from natural sources, and is globally commercially available from a wide variety of suppliers. Therefore, C-allylation of guaiacol with an allyl halide is another attractive route for preparing para-eugenol, as generally illustrated in Scheme 3.

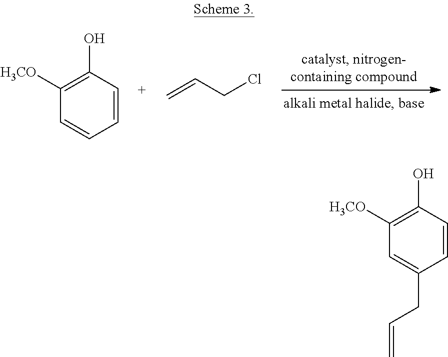

This process can be achieved in a one-pot reaction, e.g., by: a) providing a first mixture comprising a catalytic amount of a Group IB metal salt and a nitrogen-containing compound; b) cooling the first mixture to about 0° C. to about 15° C.; c) adding about 0.8 to about 1 equivalent of an alkali metal halide to the first mixture to form a second mixture; d) adding an aqueous solution of about 0.7 to about 1 equivalent of a base to the second mixture to form a third mixture; e) adding 1 equivalent of guaiacol to the third mixture dropwise to form a fourth mixture; f) allowing the fourth mixture to warm to a temperature of about 15° C. to about 25° C.; and g) adding about 1.0 to about 1.5 equivalents of an allyl halide to the fourth mixture, maintaining a temperature of less than about 30° C., to form a fifth mixture and thereby produce para-eugenol.

In this process, all amounts described as equivalents are intended to refer to molar equivalents, with the number of moles of guaiacol being one equivalent.

Step a) of the process comprises providing a first mixture comprising a catalytic amount of a Group IB metal salt and a nitrogen-containing compound.

Group IB metals include copper, silver and gold. The Group IB metal salt may comprise any suitable anion, such as a halide (e.g., chloride, bromide or iodide). A suitable Group IB metal salt is copper(I) chloride.

The catalytic amount of the Group IB metal salt may be about 0.001% to about 10% based on the number of moles of guaiacol in the reaction. For example, the catalytic amount may be about 0.01% to about 8% or about 0.1% to about 5%, or about: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% based on the number of moles of guaiacol in the reaction.

The first mixture further includes a nitrogen-containing compound, which may be at least one of ammonia and salts thereof, an amine, an amino acid, an amide, a hydrazine, and a basic cross-linked polystyrene resin. The ammonia salt may be any suitable salt such as ammonium chloride, bromide, phosphate, acetate, molybdate, or the like. The amine may be an aliphatic amine or a diamine including primary, secondary and tertiary amines. The amino acid can be any suitable amino acid, such as a naturally occurring amino acid such as glycine. The hydrazine may be hydrazine itself or a salt thereof, or a substituted hydrazine. A suitable nitrogen-containing compound is aqueous ammonia.

In some embodiments, the mixture of the Group IB metal salt and the nitrogen-containing compound may be provided in water. The nitrogen-containing compound may form a complex with the catalytic Group IB metal in the first mixture.

Step b) of the process comprises cooling the first mixture to about 0° C. to about 15° C., or 5° C. to 12° C. For example, step b) may comprise cooling the first mixture to about: 0° C., 1° C., 2° C., 3° C., 4° C., 5° C., 6° C., 7° C., 8° C., 9° C., 10° C., 11° C., 12° C., 13° C., 14° C., or 15° C. In some embodiments, step b) comprises cooling the first mixture to about 10° C.

Step c) of the process comprises adding about 0.8 to about 1 equivalent of an alkali metal halide to the first mixture to form a second mixture. The alkali metal may be any suitable alkali metal such as lithium, sodium or potassium, and the halide may be any suitable halide such as chloride, bromide or iodide. In some embodiments, the alkali metal halide may comprise sodium iodide.

In some embodiments, step c) comprises adding 0.80 to 1.00 equivalents, or 0.85 to 0.95 equivalents, e.g., about: 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.00 equivalents of the alkali metal halide to the first mixture to form the second mixture.

In some embodiments, step c) further comprises maintaining a temperature of less than about 20° C. during the addition of the alkali metal halide. For example, step c) may comprise maintaining a temperature of less than about: 20° C., 19° C., 18° C., 17° C., 16° C., 15° C., 14° C., 13° C., 12° C., 11° C., 10° C., 9° C., 8° C., 7° C., 6° C., or 5° C. In some embodiments, step c) comprises maintaining a temperature of less than about 15° C. during the addition of the alkali metal halide.

Step d) of the process comprises adding an aqueous solution of about 0.7 to about 1 equivalent of a base to the second mixture to form a third mixture. The base may be any suitable base, such as an inorganic base. A suitable base may be an alkali or alkaline earth metal salt of a hydroxide, carbonate, bicarbonate or acetate. In some embodiments, the base may comprise sodium hydroxide.

In some embodiments, step c) comprises adding about 0.70 to 1.00 equivalents, or 0.80 to 0.95 equivalents, or 0.85 to 0.90 equivalents, e.g., 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.00 equivalents of the base to the second mixture to form the third mixture.

Step e) of the process comprises adding 1 equivalent of guaiacol to the third mixture dropwise to form a fourth mixture.

Step f) of the process comprises allowing the fourth mixture to warm to a temperature of about 15° C. to about 25° C. For example, step f) may comprise allowing the fourth mixture to warm to a temperature of about: 15° C., 16° C., 17° C., 18° C., 19° C., 20° C., 21° C., 22° C., 23° C., 24° C., or 25° C. In some embodiments, step f) of the process comprises allowing the fourth mixture to warm to a temperature of about 20° C. to about 22° C. The fourth mixture may be allowed to warm by adding heat to the mixture, or by simply removing any source of cooling and thereby allowing the mixture to equilibrate to the appropriate temperature, such as room temperature.

Step g) of the process comprises adding about 1.0 to about 1.5 equivalents of an allyl halide to the fourth mixture, maintaining a temperature of less than about 30° C., to form a fifth mixture and thereby produce para-eugenol.

The allyl halide may be any suitable allyl halide such as allyl chloride, allyl bromide or allyl iodide. In a suitable embodiment, the allyl halide may be allyl chloride.

Step g) comprises maintaining a temperature of less than about 30° C. For example, step g) may comprise maintaining a temperature of less than about: 30° C., 29° C., 28° C., 27° C., 26° C., 25° C., 24° C., 23° C., 22° C., 21° C., 20° C., 19° C., 18° C., 17° C., 16° C., or 15° C. In a suitable embodiment, step g) comprises maintaining a temperature of between about 20° C. and about 25° C.

In some embodiments, step g) may further comprise stirring the mixture for about 5 minutes to about 15 minutes. For example, step g) may comprise stirring the mixture for 5 to 15 minutes, or 8 to 12 minutes, e.g., about: 5, 6, 7, 8, 9, 10, 11, 12, 13, 14, or 15 minutes.

Following step g), the process may further comprise various steps to purify the para-eugenol. For example, the fifth mixture may be neutralized by adding a mineral acid to form a sixth mixture. The mineral acid may be any suitable mineral acid, such as an aqueous solution of hydrochloric acid. The mineral acid may be added dropwise, and the temperature may be maintained at about 25° C. during addition of the acid.

The sixth mixture may be extracted with an organic solvent to provide an organic extract. The organic solvent may be a polar or non-polar solvent. For example, the organic solvent may be diethyl ether, chloroform, methylene chloride, ethyl acetate, benzene, toluene, pentane, hexane, cyclohexane, 1,2-dichloroethane, or any mixture thereof. In some embodiments, the organic solvent may be diethyl ether. The organic extract may be dried to remove any residual water. For example, the organic extract may be dried over sodium sulfate or magnesium sulfate, and decanted or filtered. The solvent may then be removed to provide a crude product.

If desired, the crude product may be further purified. For example, the crude product may be dissolved in a basic aqueous solution, such as an aqueous solution of a base such as an inorganic base (e.g., sodium hydroxide). This solution may be extracted with an organic solvent to remove any non-phenolic compounds, and provide an extracted aqueous solution. The organic solvent may be a polar or non-polar solvent. For example, the organic solvent may be diethyl ether, chloroform, methylene chloride, ethyl acetate, benzene, toluene, pentane, hexane, cyclohexane, 1,2-dichloroethane, or any mixture thereof. In some embodiments, the organic solvent may be diethyl ether.

The extracted aqueous solution may then be neutralized with an acid, such as an aqueous solution of a mineral acid (e.g., aqueous hydrochloric acid). The neutralized solution may then be extracted with an organic solvent to provide a second organic extract. Again, the organic solvent may be a polar or non-polar solvent, such as diethyl ether, chloroform, methylene chloride, ethyl acetate, benzene, toluene, pentane, hexane, cyclohexane, 1,2-dichloroethane, or any mixture thereof. The second organic extract may be dried to remove any residual water, e.g., using sodium sulfate or magnesium sulfate, and decanted or filtered. The solvent may then be removed to provide a final product mixture.

The final product mixture can be further purified to separate any final products. For example, the final product mixture can be purified using distillation (e.g., high vacuum distillation), column chromatography, freeze crystallization, adduct crystallization with phenolics or amines, derivatization such as acetyl derivatization and the like, or any combination thereof.

The process may produce para-eugenol as the major product. The process may also produce a smaller amount of ortho-eugenol as a byproduct. For example, the final product may comprise about 70-95%, 75-90% or 80-85% para-eugenol and about 5-30%, 10-25% or 15-20% ortho-eugenol. For example, the final product may comprise about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94% or 95% para-eugenol, or more. The process may also produce ortho-eugenol as a minor product. For example, the final product may comprise about: 30%, 29%, 29%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, or 5% ortho-eugenol, or less.

The process may further comprise synthesizing a eugenol end-capped polydiorganosiloxane, such as a eugenol end-capped polydimethylsiloxane, using the product comprising para-eugenol. The product comprising para-eugenol may be purified, or it may include some ortho-eugenol as described above. The eugenol end-capped polydimethylsiloxane may be synthesized using any means known in the art, such as a hydrosilylation reaction. Suitable methods are described above and herein.

Disclosed are polysiloxane-polycarbonate copolymers. The polysiloxane-polycarbonate copolymers have repeating units of both polycarbonate and polysiloxane structural units.

The polycarbonate structural unit of the polysiloxane-polycarbonate copolymer may have repeating units of formula (1):

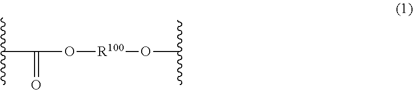

wherein each R¹⁰⁰ may independently comprise any suitable organic group, such as an aliphatic, alicyclic, or aromatic group, or any combination thereof. In certain embodiments, R¹⁰⁰ in the carbonate units of formula (1) may be a C₆-C₃₆ aromatic group wherein at least one moiety is aromatic.

The repeating units of formula (1) may be derived from dihydroxy compounds of formula (2):

HO—R¹⁰⁰—OH (2)

wherein R¹⁰⁰ is as defined above.

The polycarbonate may include repeating units of formula (3):

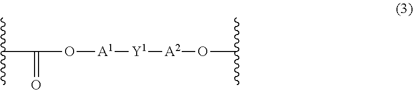

wherein each of the A¹ and A² is a monocyclic divalent aryl group and Y¹ is a bridging group having one or two atoms that separate A¹ and A². For example, one atom may separate A¹ from A², with illustrative examples of these groups including —O—, —S—, —S(O)—, —S(O)₂—, —C(O)—, methylene, cyclohexyl-methylene, 2-[2.2.1]-bicycloheptylidene, ethylidene, isopropylidene, neopentylidene, cyclohexylidene, cyclopentadecyclidene, cyclododecylidene, and adamantylidene. The bridging group of Y¹ may be a hydrocarbon group such as methylene, cyclohexylidene, or isopropylidene.

The repeating units of formula (3) may be derived from a dihydroxy monomer unit of formula (4):

HO-A¹-Y¹-A²-OH (4)

wherein A¹, A², and Y¹ are as defined above.

The polycarbonate may include repeating units of formula (5):

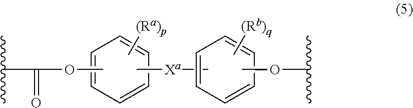

wherein R^{a} and R^{b} are each independently halogen, C₁-C₁₂ alkyl, C₁-C₁₂ alkenyl, C₃-C₈ cycloalkyl, or C₁-C₁₂ alkoxy; p and q are each independently 0 to 4; and X^{a} is a bridging group between the two arylene groups. X^{a} may be a single bond, —O—, —S—, —S(O)—, —S(O)₂—, —C(O)—, or a C₁-C₁₈ organic group. The C₁-C₁₈ organic bridging group may be cyclic or acyclic, aromatic or non-aromatic, and can optionally include halogens, heteroatoms (e.g., oxygen, nitrogen, sulfur, silicon, or phosphorous), or a combination thereof. The C₁-C₁₈ organic group can be disposed such that the C₆ arylene groups connected thereto are each connected to a common alkylidene carbon or to different carbons of the C₁-C₁₈ organic bridging group. The bridging group X^{a} and the carbonate oxygen atoms of each C₆ arylene group can be disposed ortho, meta, or para (specifically para) to each other on the C₆ arylene group. Exemplary X^{a} groups include, but are not limited to, methylene, ethylidene, neopentylidene, isopropylidene, cyclohexylmethylidene, 1,1-ethene, 2-[2.2.1]-bicycloheptylidene, cyclohexylidene, cyclopentylidene, cyclododecylidene, and adamantylidene.

In certain embodiments, p and q are each 1; R^{a} and R^{b} are each a C₁-C₃ alkyl group, specifically methyl, disposed meta to the oxygen on each ring; and X^{a} is isopropylidene. In certain embodiments, p and q are both 0; and X^{a} is isopropylidene.

In certain embodiments, X^{a} may have formula (6):

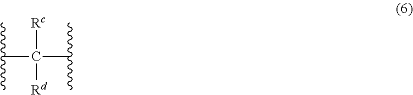

wherein R^{c} and R^{d} are each independently hydrogen, halogen, alkyl (e.g., C₁-C₁₂ alkyl), cycloalkyl (e.g., C₃-C₁₂ cycloalkyl), cycloalkylalkyl (e.g., C₃-C₁₂-cycloalkyl-C₁-C₆-alkyl), aryl (e.g., C₆-C₁₂ aryl), arylalkyl (e.g., C₆-C₁₂-aryl-C₁-C₆-alkyl), heterocyclyl (e.g., five- or six-membered heterocyclyl having one, two, three, or four heteroatoms independently selected from nitrogen, oxygen, and sulfur), heterocyclylalkyl (e.g., five- or six-membered heterocyclyl-C₁-C₆-alkyl), heteroaryl (e.g., five- or six-membered heteroaryl having one, two, three, or four heteroatoms independently selected from nitrogen, oxygen, and sulfur), or heteroarylalkyl (e.g., five- or six-membered heteroaryl-C₁-C₆-alkyl), wherein said alkyl, cycloalkyl, cycloalkylalkyl, aryl, arylalkyl, heterocyclyl, heterocyclylalkyl, heteroaryl, and heteroarylalkyl are each independently unsubstituted or substituted (e.g., substituted with 1 to 3 substituents independently selected from the group consisting of —OH, —NH₂, —NO₂, —CN, halo, C₁-C₄-alkyl, C₁-C₄-alkoxy-C₁-C₄-alkyl, halo-C₁-C₄-alkyl, halo-C₁-C₄-alkoxy-C₁-C₄-alkyl, hydroxy-C₁-C₄-alkyl, amino-C₁-C₄-alkyl, C₁-C₄-alkylamino-C₁-C₄-alkyl, di(C₁-C₄-alkyl)amino-C₁-C₄-alkyl, azido-C₁-C₄-alkyl, cyano-C₁-C₄-alkyl, C₁-C₄-alkoxy, halo-C₁-C₄-alkoxy, C₁-C₄-alkoxy-C₁-C₄-alkoxy, C₂-C₄-alkenyl, and C₂-C₄-alkynyl). In certain embodiments, R^{c} and R^{d} are each independently hydrogen or C₁-C₈ alkyl. In certain embodiments, R^{c} and R^{d} are each methyl. Exemplary groups of formula (6) include, but are not limited to, methylene, ethylidene, neopentylidene, and isopropylidene.

In certain embodiments, X^{a} may have formula (7):

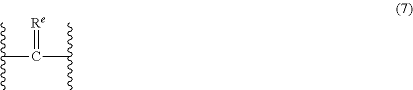

wherein R^{e} is a divalent C₁-C₃₁ group. In certain embodiments, R^{e} is a divalent hydrocarbyl (e.g., a C₁₂-C₃₁ hydrocarbyl), a cycloalkylidene (e.g., a C₅-C₁₈ cycloalkylidene), a cycloalkylene (e.g., a C₅-C₁₈ cycloalkylene), a heterocycloalkylidene (e.g., a C₃-C₁₈ heterocycloalkylidene), or a group of the formula —B¹-G-B²— wherein B¹ and B² are the same or different alkylene group (e.g., a C₁-C₆ alkylene group) and G is a cycloalkylidene group (e.g., a C₃-C₁₂ cycloalkylidene group) or an arylene group (e.g., a C₆-C₁₆ arylene group), wherein said hydrocarbyl, cycloalkylidene, cycloalkylene, and heterocycloalkylidene are each independently unsubstituted or substituted (e.g., substituted with 1 to 3 substituents independently selected from the group consisting of —OH, —NH₂, —NO₂, —CN, halo, C₁-C₄-alkyl, C₁-C₄-alkoxy-C₁-C₄-alkyl, halo-C₁-C₄-alkyl, halo-C₁-C₄-alkoxy-C₁-C₄-alkyl, hydroxy-C₁-C₄-alkyl, amino-C₁-C₄-alkyl, C₁-C₄-alkylamino-C₁-C₄-alkyl, di(C₁-C₄-alkyl)amino-C₁-C₄-alkyl, azido-C₁-C₄-alkyl, cyano-C₁-C₄-alkyl, C₁-C₄-alkoxy, halo-C₁-C₄-alkoxy, C₁-C₄-alkoxy-C₁-C₄-alkoxy, C₂-C₄-alkenyl, and C₂-C₄-alkynyl). Exemplary groups of formula (7) include, but are not limited to, 2-[2.2.1]-bicycloheptylidene, cyclohexylidene, cyclopentylidene, cyclododecylidene, and adamantylidene.

The repeating structural units of formula (5) may be derived from a dihydroxy monomer unit of formula (8):

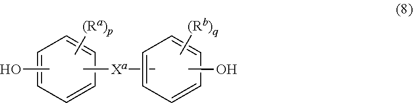

wherein X^{a}, R^{a}, R^{b}, p, and q are as defined above. In certain embodiments, p and q are both 0, and X^{a} is isopropylidene.

Exemplary monomers for inclusion in the polycarbonate include, but are not limited to, 4,4′-dihydroxybiphenyl, 1,1-bis(4-hydroxyphenyl)methane, bis(4-hydroxyphenyl)acetonitrile, bis(4-hydroxyphenyl)phenylmethane, bis(4-hydroxyphenyl)-1-naphthylmethane, 1,1-bis(4-hydroxyphenyl)ethane, 1,2-bis(4-hydroxyphenyl)ethane, 1,1-bis(4-hydroxyphenyl)-1-phenylethane, 1,1-dichloro-2,2-bis(4-hydroxyphenyl)ethylene, 1,1-dibromo-2,2-bis(4-hydroxyphenyl)ethylene, 1,1-dichloro-2,2-bis(5-phenoxy-4-hydroxyphenyl)ethylene, 1,1-bis(4-hydroxyphenyl)propane, 1,1-bis(4-hydroxy-t-butylphenyl)propane, 2,2-bis(4-hydroxyphenyl)propane (“bisphenol-A” or “BPA”), 2-(4-hydroxyphenyl)-2-(3-hydroxyphenyl)propane, 2,2-bis(4-hydroxy-2-methylphenyl)propane, 2,2-bis(3-methyl-4-hydroxyphenyl)propane, 2,2-bis(3-ethyl-4-hydroxyphenyl)propane, 2,2-bis(3-n-propyl-4-hydroxyphenyl)propane, 2,2-bis(3-isopropyl-4-hydroxyphenyl)propane, 2,2-bis(3-sec-butyl-4-hydroxyphenyl)propane, 2,2-bis(3-t-butyl-4-hydroxyphenyl)propane, 2,2-bis(3-cyclohexyl-4-hydroxyphenyl)propane, 2,2-bis(3-allyl-4-hydroxyphenyl)propane, 2,2-bis(3-methoxy-4-hydroxyphenyl)propane, 2,2-bis(4-hydroxy-3-bromophenyl)propane, 2,2-bis(4-hydroxyphenyl)hexafluoropropane, 1,1-bis(4-hydroxyphenyl)n-butane, 2,2-bis(4-hydroxyphenyl)butane, 3,3-bis(4-hydroxyphenyl)-2-butanone, 1,1-bis(4-hydroxyphenyl)isobutene, trans-2,3-bis(4-hydroxyphenyl)-2-butene, 1,6-bis(4-hydroxyphenyl)-1,6-hexanedione, 2,2-bis(4-hydroxyphenyl)octane, 1,1-bis(hydroxyphenyl)cyclopentane, 1,1-bis(4-hydroxyphenyl)cyclohexane, 1,1-bis(4-hydroxy-3-methylphenyl)cyclohexane, 1,1-bis(4-hydroxyphenyl)cyclododecane, 2,2-bis(4-hydroxyphenyl)adamantane, (alpha, alpha′-bis(4-hydroxyphenyl)toluene, 4,4′-dihydroxybenzophenone, 2,7-dihydroxypyrene, bis(4-hydroxyphenyl)ether, ethylene glycol bis(4-hydroxyphenyl)ether, bis(4-hydroxyphenyl) sulfide, bis(4-hydroxyphenyl)sulfoxide, bis(4-hydroxyphenyl)sulfone, bis(4-hydroxyphenyl)diphenylmethane, 1,6-dihydroxynaphthalene, 2,6-dihydroxynaphthalene, 6,6′-dihydroxy-3,3,3′,3′-tetramethylspiro(bis)indane (“spirobiindane bisphenol”), 2,6-dihydroxydibenzo-p-dioxin, 2,6-dihydroxythianthrene, 2,7-dihydroxyphenoxathin, 2,7-dihydroxy-9,10-dimethylphenazine, 3,6-dihydroxydibenzofuran, 3,6-dihydroxydibenzothiophene, 2,7-dihydroxycarbazole, 2-phenyl-3,3-bis(4-hydroxyphenyl)phthalimidine (also referred to as 3,3-bis(4-hydroxyphenyl)-2-phenylisoindolin-1-one or “PPPBP”), 9,9-bis(4-hydroxyphenyl)fluorene, and bisphenol isophorone (also referred to as 4,4′-(3,3,5-trimethylcyclohexane-1,1-diyl)diphenol or “BPI”), 1,1-bis(4-hydroxy-3-methylphenyl)cyclohexane (“DMBPC”), tricyclopentadienyl bisphenol (also referred to as 4,4′-(octahydro-1H-4,7-methanoindene-5,5-diyl)diphenol), 2,2-bis(4-hydroxyphenyl)adamantane (“BCF”), 1,1-bis(4-hydroxyphenyl)-1-phenyl ethane (“BPAP”), and 3,3-bis(4-hydroxyphenyl)phthalide, or any combination thereof.

The polysiloxane structural units of the polysiloxane-polycarbonate copolymer may have repeating units of formula (9):

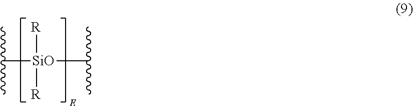

wherein each R is independently a C₁-C₁₃ monovalent organic group. For example, R can be a C₁-C₁₃ alkyl, C₁-C₁₃ alkoxy, C₂-C₁₃ alkenyl, C₂-C₁₃ alkenyloxy, C₃-C₆ cycloalkyl, C₃-C₆ cycloalkoxy, C₆-C₁₄ aryl, C₆-C₁₀ aryloxy, C₇-C₁₃ arylalkyl, C₇-C₁₃ aralkoxy, C₇-C₁₃ alkylaryl, or C₇-C₁₃ alkylaryloxy. The foregoing groups can be fully or partially halogenated with fluorine, chlorine, bromine, or iodine, or a combination thereof. Where a transparent poly(carbonate-siloxane) is desired, R is unsubstituted by halogen. Combinations of the foregoing R groups can be used in the same copolymer.

The value of E in formula (9) can vary widely depending on the type and relative amount of each component in the composition, the desired properties of the composition, and like considerations. Generally, E has an average value of 1 to 1,000, specifically 2 to 500, 2 to 200, 10 to 200, 2 to 125, 5 to 80, 10 to 100, 10 to 70, 30 to 60, or 40 to 50. E may have an average value of 10 to 100, 10 to 80, 10 to 40, 40 to 80, 30 to 60, 40 to 70, or 40 to 50. Where E is of a lower value (e.g., less than 40), it can be desirable to use a relatively larger amount of the poly(carbonate-siloxane). Conversely, where E is of a higher value (e.g., greater than 40), a relatively lower amount of the poly(carbonate-siloxane) can be used. A combination of a first and a second (or more) poly(carbonate-siloxane) can be used, wherein the average value of E of the first copolymer is less than the average value of E of the second copolymer.

The polysiloxane blocks may be provided by repeating structural units of formula (10):

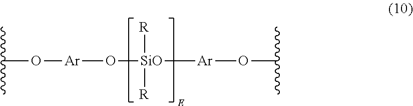

wherein E and R are as defined in formula (9), and each Ar is independently a substituted or unsubstituted C₆-C₃₀ arylene wherein the bonds are directly connected to an aromatic moiety. The Ar groups in formula (10) can be derived from a C₆-C₃₀ dihydroxyarylene compound, for example a dihydroxyarylene compound of formula (2), (4), or (8) above.

In a preferred embodiment, the polysiloxane blocks are of formula (11):

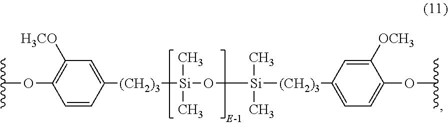

wherein E has an average value of 1 to 1000, 2 to 200, 10 to 200, 2 to 125, 5 to 125, 1 to 100, 5 to 100, 10 to 100, 5 to 50, 20 to 80, 30 to 60, 40 to 50, or 5 to 20.

Polysiloxane blocks of formula (11) can be derived from the corresponding dihydroxy polysiloxane of formula (12):

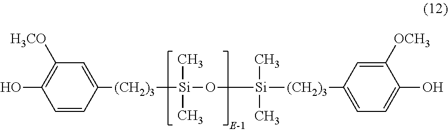

wherein E is as described for formula (11). Such dihydroxy polysiloxanes can be made by affecting a platinum-catalyzed addition between a siloxane hydride and eugenol.

The polysiloxane-polycarbonate copolymers may comprise carbonate units of formula (1) derived from bisphenol A and polysiloxane units as described above, in particular polysiloxane units of formula (11), wherein E has an average value of 1 to 1000, specifically 10 to 100, more specifically 30 to 60, more specifically 40 to 60, and still more specifically 40 to 50. The polysiloxane-polycarbonate copolymer can comprise the siloxane units in an amount of 0.1 to 60 weight percent (wt %), 0.5 to 55 wt %, 0.5 to 45 wt %, 0.5 to 30 wt %, 2.0 to 30 wt %, or 0.5 to 20 wt %, based on the total weight of the polycarbonate copolymer, with the proviso that the siloxane units are covalently bound to the polymer backbone of the polycarbonate copolymer.

In an embodiment, the polysiloxane-polycarbonate copolymers are of formula (13):

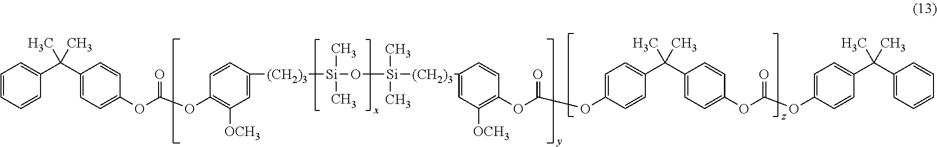

wherein x is 1 to 1000, 1 to 200, 20 to 200, 10 to 200, specifically 5 to 85, specifically 10 to 70, specifically 15 to 65, 30 to 60, and more specifically 40 to 50; y is 1 to 500, or 10 to 200, and z is 1 to 1000, or 10 to 800. In an embodiment, x is 1 to 200, y is 1 to 90 and z is 1 to 600, and in another embodiment, x is 30 to 50, y is 10 to 30 and z is 45 to 600. The polysiloxane blocks may be randomly distributed or controlled distributed among the polycarbonate blocks.

The polysiloxane-polycarbonate copolymer, such as a polydimethylsiloxane-polcarbonate copolymer, may include 1 wt % to 35 wt % siloxane content (e.g., polydimethylsiloxane content), 1 wt % to 30 wt % siloxane content, 2 wt % to 30 wt % siloxane content, 1 wt % to 25 wt % siloxane content, 5 wt % to 25 wt % siloxane content, 6 wt % to 20 wt % siloxane content, or 3 wt % to 8 wt % siloxane content. The polysiloxane-polycarbonate copolymer, such as a polydimethylsiloxane-polcarbonate copolymer, may include 1 wt % to 35 wt %, or 1 wt % to 10 wt %, or 15 wt % to 25 wt % silioxane content, e.g., about: 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, or about 35 wt % siloxane content. The polysiloxane-polycarbonate copolymer may include about 6 wt % siloxane content. The polysiloxane-polycarbonate copolymer may include about 20 wt % siloxane content. Siloxane content may refer to polydimethylsiloxane content.

The polysiloxane-polycarbonate copolymer may have a weight average molecular weight (Mw) of 17,000 g/mol to 50,000 g/mol, 17,000 g/mol to 40,000 g/mol, 18,000 g/mol to 40,000 g/mol, 17,000 g/mol to 35,000 g/mol, 20,000 g/mol to 35,000 g/mol, 23,000 g/mol to 30,000 g/mol, or 22,000 g/mol to 24,000 g/mol. The polysiloxane-polycarbonate copolymer may have a weight average molecular weight (Mw) of 17,000 to 40,000 g/mol, 20,000 to 35,000 g/mole, e.g., about: 17,000 g/mol, about 17,500 g/mol, about 18,000 g/mol, about 18,500 g/mol, about 19,000 g/mol, about 19,500 g/mol, about 20,000 g/mol, about 20,500 g/mol, about 21,000 g/mol, about 21,500 g/mol, about 22,000 g/mol, about 22,500 g/mol, about 23,000 g/mol, about 23,500 g/mol, about 24,000 g/mol, about 24,500 g/mol, about 25,000 g/mol, about 25,500 g/mol, about 26,000 g/mol, about 26,500 g/mol, about 27,000 g/mol, about 27,500 g/mol, about 28,000 g/mol, about 28,500 g/mol, about 29,000 g/mol, about 29,500 g/mol, about 30,000 g/mol, about 30,500 g/mol, about 31,000 g/mol, about 31,500 g/mol, about 32,000 g/mol, about 32,500 g/mol, about 33,000 g/mol, about 33,500 g/mol, about 34,000 g/mol, about 34,500 g/mol, about 35,000 g/mol, about 35,500 g/mol, about 36,000 g/mol, about 36,500 g/mol, about 37,000 g/mol, about 37,500 g/mol, about 38,000 g/mol, about 38,500 g/mol, about 39,000 g/mol, about 39,500 g/mol, or about 40,000 g/mol. The polysiloxane-polycarbonate copolymer may have a weight average molecular weight of about 23,000 g/mol, or about 30,000 g/mol. Weight average molecular weight can be determined by gel permeation chromatography (GPC) using BPA polycarbonate standards.

The polysiloxane-polycarbonate copolymer may have a polysiloxane average block length of 1 to 1000 units. The polysiloxane-polycarbonate copolymer may have a polysiloxane average block length of 10 to 200 units. The polysiloxane-polycarbonate copolymer may have a polysiloxane average block length of 10 to 100 units. The polysiloxane-polycarbonate copolymer may have a polysiloxane average block length of 30 to 100 units. The polysiloxane-polycarbonate copolymer may have a polysiloxane average block length of 30 to 60 units. The polysiloxane-polycarbonate copolymer may have a polysiloxane average block length of 40 to 60 units. The polysiloxane-polycarbonate copolymer may have a polysiloxane average block length of 40 to 50 units. The polysiloxane-polycarbonate copolymer may have a polysiloxane average block length of 45 units.

Disclosed is a method to prepare polysiloxane-polycarbonate copolymer.

One of the reagents employed is an oligomeric aromatic polycarbonate. The structural units in said oligomeric polycarbonate may all have the same structure or may have differing structures; i.e., the oligomeric polycarbonate may be a copolycarbonate. The structural units of the oligomeric polycarbonate may comprise carbonate units of formula (1) derived from bisphenol A, but it should be understood that other dihydroxyaromatic compounds shown above may be substituted for all or part of the bisphenol when appropriate.

Oligomeric polycarbonates may be prepared by any known method for polycarbonate preparation. Included are interfacial, transesterification and redistribution methods. One often preferred method comprises contacting at least one dihydroxyaromatic compound with phosgene, the molar ratio of phosgene to dihydroxyaromatic compound being in the range of about 0.1-0.9:1, preferably about 0.3-0.85:1 and most preferably about 0.5-0.8:1, in an alkaline mixed aqueous-organic liquid at a pH in the range of about 9-11, in the presence of at least one trialkylamine as the only catalytic species present and, optionally, at least one monohydroxyaromatic compound or chloroformate thereof as chain termination agent.

Illustrative organic liquids which may be used in oligomer preparation are aliphatic hydrocarbons such as n-hexane and n-heptane; chlorinated aliphatic hydrocarbons such as methylene chloride, chloroform, carbon tetrachloride, dichloroethane, trichloroethane, tetrachloroethane, dichloropropane and 1,2-dichloroethylene; aromatic hydrocarbons such as benzene, toluene and xylene; substituted aromatic hydrocarbons such as chlorobenzene, o-dichlorobenzene, the chlorotoluenes, nitrobenzene and acetophenone; and carbon disulfide. The chlorinated aliphatic hydrocarbons, especially methylene chloride, are preferred.

The catalyst is a tertiary amine, typically a trialkylamine or a highly nucleophilic heterocyclic amine such as 4-dimethylaminomorpholine. Tertiary amine mixtures may also be employed. Among tertiary amines that can be used are aliphatic tertiary amines such as triethylamine, tributylamine, cycloaliphatic amines such as N,N-diethyl-cyclohexylamine and aromatic tertiary amines such as N,N-dimethylaniline. Triethylamine is preferred.

The oligomer-forming reaction is generally conducted at a temperature in the range of about 15-50° C. The pH of the aqueous phase of the reaction mixture is maintained in the range of about 9-12 by introduction of a suitable base, most often an alkali metal hydroxide and preferably sodium hydroxide.

A monohydroxyaromatic compound or chloroformate thereof may be present as a chain termination agent in the oligomer preparation method. Illustrative chain termination agents are phenol, p-cumylphenol and their chloroformates.

For oligomer preparation, the molar ratio of phosgene to bisphenol is conveniently maintained in the range of about 0.1-0.9:1. For preparation of a transparent product, a molar ratio of about 0.3-0.85:1 and preferably about 0.5-0.8:1 is suitable. The pH of the aqueous phase of the reaction mixture is maintained in the range of about 9-11. Tertiary amine proportions are typically in the range of about 0.01-2.0 mole percent based on bisphenol. The oligomer concentration is in the range of about 5-30% by weight, based on oligomer plus solvent. Chain termination agent, if present, may be in an amount up to about 10 mole percent based on bisphenol. As noted hereinafter, however, introduction of chain termination agent at various stages of the process is contemplated and the amount present for oligomer preparation, if any, may be substantially less than the total to be employed overall.

Molecular weights (weight average relative to polystyrene, measured by gel permeation chromatography, whenever used herein in any context) for the oligomeric polycarbonates employed are not critical. However, they will, of course, be lower than the corresponding molecular weights of commercial polycarbonates. For the most part, illustrative molecular weights may be in the range of about 1,000-8,000.

A feature of the disclosed method is the ability to achieve a low haze product using only one charge of bisphenol, which in most instances is in the form of a solid and is therefore difficult to meter into the reaction mixture on a continuous or semi-continuous basis. Thus, in a preferred embodiment all the bisphenol employed in the process is furnished by the oligomeric polycarbonate. Other reagents may be supplied at various stages of the process, but the bisphenol-derived reagent is present in its entirety at the beginning.

In an alternative embodiment, a minor proportion of total bisphenol, generally up to about 10% by weight of the total amount, may be introduced at one or more than one point subsequent to initial introduction thereof. The point or points of such introduction may be anywhere from immediately after initial bisphenol introduction to after siloxane-BCF (as defined hereinafter) introduction. A major purpose of later introduction of bisphenol is frequently to accommodate various process streams, particularly in a continuous reaction scheme.

The eugenol end-capped polydimethylsiloxane bischloroformate, hereinafter sometimes designated “siloxane-BCF” for brevity, may be all or part of a previously synthesized batch or may be prepared as needed on a just-in-time basis. Particularly in the latter event, it need not be isolated or stored and may be employed in the form in which it was prepared.

The present disclosure employs phosgene (COCl₂) to convert siloxane bisphenol OH groups into the corresponding chloroformate groups. It has been discovered that the amount of phosgene employed strongly influences product yield. Phosgene is preferably used in an amount corresponding to between about 2.5 and about 6, even more preferably between about 3.5 and about 5.5 moles of phosgene per mole of siloxane bisphenol OH group. Expressed in terms of moles of phosgene per mole of siloxane bisphenol employed, it is preferable to use between about 5 and about 12, and even more preferable between about 7 and about 11 moles of phosgene per mole of siloxane bisphenol.

The alkali metal hydroxide or alkaline earth metal hydroxide, or combination thereof is employed as an aqueous solution used in an amount preferably corresponding to between about 3.5 and about 6, and even more preferably between about 4 and about 5 moles of metal hydroxide per mole of phosgene employed. The concentration of the aqueous metal hydroxide solution employed is preferably between about 5 and about 25, and even more preferably between about 17 and about 25 percent by weight metal hydroxide. In one embodiment the concentration of the metal hydroxide solution is at least about 5 percent by weight. Of course, more concentrated solutions of metal hydroxide may be used, as long as they are supplemented with water such that the net metal hydroxide concentration in aqueous solution is about 25% by weight or less.

The siloxane bisphenol is typically introduced into the reactor as a solution in a solvent. Typically the solvent is methylene chloride but can be any solvent suitable for use under interfacial reaction conditions. Typically halogenated solvents such as methylene chloride, chloroform, and 1,2-dichloroethane are preferred but other non-halogenated solvents such as toluene or ethyl acetate may also be used. Typically the concentration of the siloxane bisphenol in the solvent is in a range between about 5 and about 95, preferably between about 10 and about 30 percent by weight siloxane bisphenol. As noted, the siloxane bisphenol employed may be a single chemical species or a mixture of chemical species as is typical in siloxane bisphenols which typically comprise a distribution of bisphenols possessing siloxane subunits of varying chain lengths. Alternatively, the siloxane bisphenol may be introduced as an oil, without solvent.

In the present disclosure the siloxane bisphenol employed comprises formula (12), wherein E is as described for formula (11).

Siloxane bisphenols may be prepared by hydrosilylation of eugenol with a siloxane dihydride in the presence of a platinum catalyst. This process is illustrated below for siloxane bisphenol (12),

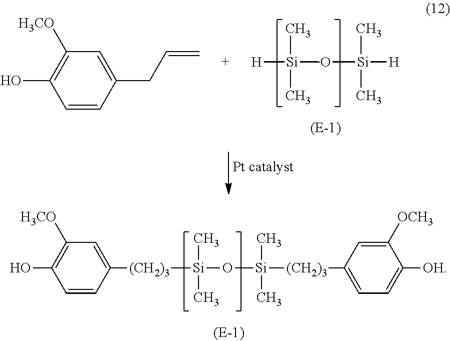

In one embodiment employing the siloxane bisphenol (12) as a reactant, E averages between 1 and about 200. In an alternate embodiment E averages between about 40 and about 50. Those skilled in the art will understand that the values given for E in formula 12 represent average values and that, for example, an E value of 45 represents a mixture of siloxane bisphenol homologues having an average value of E of about 45.

Typically, siloxane bisphenol (12), aqueous metal hydroxide, and phosgene are introduced at one or more upstream positions along the reactor. As mentioned, these reactants pass through the reactor, forming siloxane-BCF during passage from the point at which the reactants are introduced and the point at which an effluent stream containing siloxane-BCF emerges from the reactor. The time required for a reactant to travel from the point at which it is introduced to the point at which either it or a product derived from it emerges from the reactor is referred to as the residence time for the reactant. Typically, residence times for each reactant is in a range between about 5 and about 800 seconds, preferably between about 10 and about 500 seconds. Those skilled in the art will understand however that the most preferred residence time will depend upon the structure of the starting siloxane bisphenol, the type of reactor employed, and that the most preferred residence time may be determined by straightforward and limited experimentation.

In the practice of the disclosed methods at least one siloxane bisphenol (12), phosgene, and at least one alkali metal hydroxide or alkaline earth metal hydroxide are introduced into a flow reactor. The flow reactor is not particularly limited and may be any reactor system which provides for the “upstream” introduction of the reactants and the “downstream” removal of product bischloroformate. Suitable flow reactor systems include tubular reactors, continuous stirred tank reactors, loop reactors, column reactors, and combinations thereof. The flow reactor may comprise a series of flow reactor components, as for example, a series of continuous stirred tank reactors arrayed such that the effluent from a first continuous stirred tank reactor provides the input for a second continuous stirred tank reactor and so forth. Combinations of the various flow reactor components are illustrated by a first column reactor coupled to a downstream continuous stirred tank reactor where the output of the column reactor represents the feed to the continuous stirred tank reactor.

Additionally, the flow reactor used according to the method of the present disclosure may comprise flow reactor components arrayed in a parallel or network fashion, for example, as where the reactants are introduced into a parallel array of two or more tubular reactors the effluent of each of which is introduced into a single continuous stirred tank reactor. In one embodiment the flow reactor comprises a series of tubular reactors. In an alternate embodiment the flow reactor comprises a series of continuous stirred tank reactors. The reactants may be introduced into the flow reactor system through one or more feed inlets attached to the flow reactor system. Typically, it is preferred that the reactants be introduced into the flow reactor through at least three feed inlets, for example where a solution of the siloxane bisphenol in an organic solvent such as methylene chloride, aqueous alkali metal hydroxide, and phosgene are introduced through separate feed inlets at or near the upstream end of a tubular reactor. Alternative arrangements wherein one or more of the reactants is introduced through multiple feed inlets at various points along the flow reactor are also possible. Typically, the relative amounts of the reactants present in the flow reactor are controlled by the rate at which they are introduced. For example, a reactant can be introduced into the flow reactor through pumps calibrated to deliver a particular number of moles of said reactant per unit time.

The present disclosure provides a method for the preparation of siloxane-BCF (14)

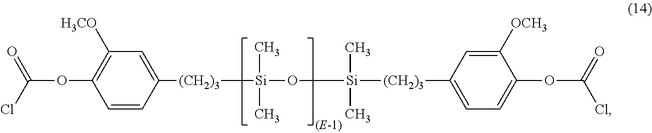

wherein E averages 1 to about 200.

Said method comprising introducing into a tubular reactor siloxane bisphenol (12), wherein E averages between 1 and about 200, as a solution in methylene chloride comprising from about 5 to about 50 weight percent siloxane bisphenol (12), an aqueous solution of sodium hydroxide, and phosgene, said phosgene being introduced at a rate such that the ratio of phosgene to siloxane bisphenol OH groups is in a range between about 2.5 and about 6 moles of phosgene per mole of siloxane bisphenol OH group, said aqueous solution of sodium hydroxide having a concentration of at least about 5 percent by weight sodium hydroxide, said aqueous solution of sodium hydroxide being introduced at a rate such that the molar ratio of metal hydroxide to phosgene is in a range between about 3.5 and about 6.

Combination of the oligomeric aromatic polycarbonate with the siloxane-BCF is generally achieved by adding the siloxane-BCF, usually in solution in an organic liquid such as those previously identified, to the aqueous-organic mixture containing the oligomeric polycarbonate in the form of either a crude reaction mixture or a purified product, while maintaining the pH of the aqueous phase in the range of about 10.5-13.5 which may be maintained by addition of aqueous base as necessary.

In one embodiment, a tubular reactor containing the siloxane-BCF reaction mixture is fed to the main resin reactor while siloxane-BCF formation is initiated.

The proportion of siloxane-BCF relative to oligomeric polycarbonate is subject to wide variation. In its broadest sense, the ratio of equivalents of oligomer hydroxide to bischloroformate, i.e., the ratio of hydroxide groups to chloroformate groups, is greater than 1:1. It is preferably at least about 4:1 and more preferably at least about 10:1. It may often be as high as about 3,000:1. To obtain a transparent product, it is generally advisable to maintain the proportion of organosiloxane units in the range of about 0.1-30.0% by weight and the value of E in the range of about 5-60.

Phosgenation of the reaction mixture and/or addition of chain termination agent can be continued during addition of the siloxane-BCF. An alternative embodiment involves interrupted phosgenation, the initial stage being during the preparation of the oligomeric aromatic polycarbonate and the subsequent stage beginning after some or all of the siloxane-BCF has been charged, with an optional delay after all of the siloxane-BCF has been introduced, said delay, when employed preferably being in the range of about 1-5 minutes. Similarly, the chain termination agent may be added in full or in part prior to or during oligomeric aromatic polycarbonate preparation or divided between such preparation and later steps. Thus, addition of both of these reagents may be continuous or performed on a programmed schedule throughout the steps of oligomeric polycarbonate and siloxane-PC oligomer preparation.

In a final step, phosgene and, optionally, chain termination agent are introduced to afford a product of a desired molecular weight. This step is most often conducted at a pH in the range of about 9.5-11.5, preferably about 10-11. Any desired molecular weight may be achieved in this step, with weight average values in the range of about 20,000 g/mol to about 100,000 g/mol being typical.

The polysiloxane-polycarbonate copolymer prepared by the method of this invention may be isolated by conventional means; for example, by anti-solvent precipitation followed by vacuum drying.

The present disclosure has multiple aspects, illustrated by the following non-limiting examples.

### EXAMPLES

### Materials

All solvents and reagents used were analytical grade. Guaiacol (greater than or equal to (≧) 98%), Allyl chloride (98%), Cu (I) chloride (≧90%), N,N-dimethyl aniline ((≧99.5%) and methyl 5-allyl-3-methoxysalicylate (97%) were purchased from Aldrich. Lithium chloride, anhydrous (99%) was purchased from SRL, aqueous ammonia (30%) was purchased from Merck. Sodium iodide (≧99.5%) was purchased from SD fine. N,N-dimethyl formamide (for HPLC, 99.8%) was purchased from Merck. 1,2-dichloroethane (≧99%), Aqueous HCl (35%) and sodium hydroxide (≧97%), sodium sulphate, anhydrous (≧99%) were purchased from Chemlab. 2% Polystyrene cross linked acidic Ion exchange resin (IER, acid meq/g of the fresh IER: 4.8-5meq/g) was purchased either from Rohm and Haas or Tulsion or Thermax.

Instrumentation:

Analysis of the reaction materials were conducted by both HPLC chromatography using C-18 reverse phase column and acetonitrile-methanol-water (0.02% H₃PO₄) as mobile phase and GC, Shimadzu GC 17A with a flame ionization detector (FID), on a HP-1 (30 meter (m)×0.53 millimeter (mm)×1.53 micrometer (μ)) column with injector and detector temperatures of 250° C. and 280° C. respectively, and a flow of 2 milliliters per minute (ml/min). Proton NMR was recorded on a Bruker 300 megaHertz (MHz) spectrometer (sample was prepared in DMSO-d6). Gas chromatography/mass spectrometry (GC/MS) was recorded on a Shimadzu 17A instrument with a mass spectrometer (MS detector).

### Example 1

### Synthesis of Para-Eugenol Via C-Allylation of Guaiacol

To 100 ml of water taken in a 4-necked RB fitted with a thermo well and a dropping funnel, CuCl (4 g) and 30% aqueous ammonia (15 milliliters (ml)) were added and the resultant dark blue complex formed was stirred for 10-15 min. Then the complex was cooled to 10° C. and NaI (120 g) was added (in batches) while maintaining the temperature <15° C. throughout the addition. After the complete addition of NaI, maintaining the temperature <15° C., aqueous solution of NaOH (32 g in 200 ml water) was added, followed by which guaiacol (100 grams (g)) was added dropwise to form a dark green mixture. The resultant reaction mixture was then warmed up to 20-22° C. and allyl chloride (72 ml) was added dropwise maintaining the temperature between 22-25° C. throughout the addition. After complete addition, the resultant mixture was stirred at the same temperature for 5 min. The mixture was then neutralized with 35% aq. HCl (60 ml), which was added dropwise while maintaining the temperature at 25° C., and then extracted with ether (5×100 ml). The combined ethereal extract was dried over anhydrous sodium sulphate and was evaporated under vacuum to obtain the crude. Conversion=87%, Selectivity of para-eugenol=60.3%.

The above mentioned crude was dissolved in aqueous NaOH and the resultant solution was extracted thrice with ether to remove all the non-phenolics. The aqueous layer thus obtained was neutralized with dilute aqueous HCl, and the resultant mixture was extracted thrice with ether to obtain the phenolics. The combined ethereal extract was dried over anhydrous sodium sulphate and was evaporated under vacuum. The resultant mixture was subjected to high vacuum distillation to obtain a fraction consisting of 85% para-eugenol and about 10% ortho-eugenol.

### Example 2

### Hydrolysis of Methyl 5-Allyl-3-Methoxysalicylate

To 55 g of methyl 5-allyl-3-methoxysalicylate taken in a 3 neck RB fitted with an overhead stirrer and a water condenser, aqueous sodium hydroxide solution (22 g of NaOH in 500 ml water) was added and the resultant mixture was refluxed for 4-5 h. Completion of reaction was monitored by HPLC (area %). After ensuring complete conversion of ester to acid, the reaction mixture was cooled to room temperature and was added drop wise into 10% dilute hydrochloric acid (70 ml 35% HCl in 175 ml water) in order to precipitate the acid. The resultant precipitate obtained was stirred for about 1 h at room temperature and then was filtered, washed thoroughly with water until chloride free and then was dried in an oven at 105° C. for 8 hours (h). 50.4 g of the crude 5-allyl-3-methoxysalicylic acid was obtained. Yield=50.4 g (97.8%), purity=99.4%.

### Example 3

### Decarboxylation of 5-allyl-3-methoxysalicylic acid

Decarboxylation of 5-allyl-3-methoxysalicylic acid was effected in a number of ways, as follows.

### Example 3a

The crude acid obtained in Example 2 (12.74 g) was taken in a two neck RB fitted with a water condenser and a nitrogen inlet. To this N,N-dimethylaniline (32 ml) was added and the resultant solution was heated to 190° C. (bath temperature) for 5-6 h. Progress and completion of the reaction was monitored by HPLC (area %). After completion of the reaction, the crude reaction mixture was subjected to high vacuum distillation in order to recover the solvent and isolate the pure product. The isolated yield of para-eugenol boiling at 104° C./4 m·bar was 8.25 g. (82.5%) with a purity of 99% (gas chromatography (GC) area %). 75% of N,N-dimethylaniline with purity of 97% was recovered.

### Example 3b

The crude acid obtained in Example 2 (25 g) was taken in a two neck RB fitted with a water condenser and a nitrogen inlet. To this N,N-dimethylaniline (5 ml) was added and the resultant solution was heated to 190° C. (bath temperature) for 5-6 h. Reaction, isolation and solvent recovery was carried out as described in example 1. The isolated yield of para-eugenol boiling at 112-116° C./13 mbar was 15.2 g. (78%) with a purity of 99.25% (refers to gas chromatography area percent (GC area %)).

### Example 3c

The crude acid obtained in Example 2 (1.0 g) was taken in a two neck RB fitted with a water condenser and a nitrogen inlet. To this N,N-dimethylaniline (0.21 ml) and lithium chloride (20 mg) were added and the resultant solution was heated to 190° C. (bath temperature) for 2 h. At the end of 2 h, the reaction mixture was quenched with dilute HCl and the organics were extracted with diethyl ether and were analyzed by HPLC. Conversion was ˜100% and the purity of crude para-eugenol was 95.5% (HPLC area %).

### Example 3d

The crude acid obtained in Example 2 (2.0 g) was taken in a two neck RB fitted with a water condenser and a nitrogen inlet. To this N,N-dimethylaniline (0.42 ml) and lithium chloride (4 mg) were added and the resultant solution was heated to 190° C. (bath temperature) for 2 h. At the end of 2 h, the reaction mixture was quenched with dilute HCl and the organics were extracted with diethyl ether and were analyzed by HPLC. Conversion was about 100% and the purity of crude para-eugenol was 97.8% (HPLC area %).

### Example 3e

The crude acid obtained in Example 2 (12.5 g) was dissolved in N,N-dimethylformamide (17.5 ml) and the resultant mixture was refluxed for 20 h. Progress and completion of the reaction was monitored by HPLC (area %). After completion of the reaction, the crude reaction mixture was subjected to high vacuum distillation in order to recover the solvent and isolate the pure product. The isolated yield of para-eugenol boiling at 104° C./4 m·bar was 8.0 g. (80.0%) with a purity of 99.7% (GC area %). 75% of N,N-dimethylformamide with purity of 99.7% was recovered.

### Example 3f

The crude acid obtained in Example 2 (50 g) was dissolved in N,N-dimethylformamide (86 ml) and the resultant mixture was refluxed for 30 h. Progress and completion of the reaction was monitored by HPLC (area %). After completion of the reaction, the crude reaction mixture was subjected to high vacuum distillation in order to recover the solvent and isolate the pure product. The isolated yield of para-eugenol boiling at 134-140° C./24 m·bar was 34.0 g. (88.0%) with a purity of 99.25% (GC area %). 82% of N,N-dimethylformamide with purity of 99.28% was recovered.

### Example 3g

The crude acid obtained in Example 2 (1.0 g) was mixed uniformly with Ca(OH)₂ (10 milligrams (mg), 1% by weight of acid) and the resultant mixture was heated at a temperature of 150-180° C. for 5-6 h. Progress and completion of the reaction was monitored by HPLC (area %). Conversion was 93% (HPLC area %) and purity of crude para-eugenol was 77% (HPLC area %).

### Example 4

### Larger-Scale Preparation of Para-Eugenol from Methyl 5-allyl-3-methoxysalicylate

Hydrolysis:

110 g of methyl 5-allyl-3-methoxysalicylate (97% pure Aldrich grade) was transferred into a 2000 ml 3-necked round bottom flask (RBF) fitted with an overhead stirrer and a reflux condenser. To this aqueous NaOH solution (44 g of NaOH in 1000 ml water) was added and the resultant mixture was refluxed with stirring (bath temp. 110° C.) for 2-3 h. Progress of the reaction was monitored by quenching aliquot sample with dilute hydrochloric acid followed by extraction with solvent (ethylene dichloride (EDC) or ether) and analyzing by HPLC (area %) for the conversion of ester to acid. Having ensured the complete conversion by HPLC, cool the reaction mixture to room temperature (25° C.) and add the same drop wise into a beaker containing dilute HCl (140 ml 35% HCl in 350 ml water) with stirring. The acid (product) precipitates as a solid and the resultant slurry was stirred for about an hour, then filtered, washed with DM water till chloride free and dried (110° C. for 8 h, moisture ˜<1%). Yield: 101 g (98%), purity: 99.7% (HPLC area %).

Decarboxylation:

To a 500 ml 3 necked RBF equipped with an overhead stirrer, N₂ inlet and a water condenser, were charged 5-allyl-3-methoxysalicylic acid (100 g, 0.48 mol., obtained above), N,N-dimethylaniline (20 ml, SRL 99.5% pure) and anhydrous lithium chloride (0.750 g, 0.0176 mol., SRL 99% pure). The above mixture was then heated gradually to 190° C. The mixture begins to melt slowly as the bath temperature reaches 130° C., becomes stirrable slurry at 150° C. and eventually a homogeneous liquid as the temperature approaches 190° C. The reaction mixture was maintained at 190° C. for about 3 h. Progress of the reaction was monitored by quenching aliquot sample with dilute hydrochloric acid followed by extraction with solvent (EDC or ether) and analyzing by HPLC (area %) for the conversion of acid to eugenol. Having ensured the complete conversion of acid by HPLC, the reaction mixture was cooled to room temperature (25° C.) and diluted with 200 ml of 1,2-dichloroethane. To this dil. HCl (28 ml of 35% HCl in 67 ml of water) was added such that the temperature of the reaction mixture does not shoot beyond 25° C. The resultant mixture was then stirred for 1 h at room temperature followed by separation of the organic and aqueous layer. The resultant organic layer was passed through a bed of acidic sulphonated polystyrene resin cross linked with divinyl benzene (commonly known as Ion exchange resin, IER) to remove the residual N,N-dimethylaniline (Spec: <2 parts per million by weight (ppm).

The solvent was distilled under vacuum (recovered) and the resultant dark brown liquid was purified by high vacuum distillation to give 71 g (Yield: 90%) of pure para-eugenol (distilled at 112-116° C. at −10-12 mbar) with purity 99.7%.

### Example 5

### Decarboxylation of methyl 5-allyl-3-methoxysalicylate

### Example 5a

To a solution of the ester (1.0 g) in N,N-dimethylformamide (2 ml), taken in a 2 necked RB fitted with a water condenser and a nitrogen inlet, lithium chloride (0.190 g) was added and the reaction mixture was heated to reflux for 12 h. At the end of 12 h the reaction mixture was quenched with water and the organics were extracted with diethyl ether and were analyzed by HPLC. Conversion was 98% and the purity of crude para-eugenol was 93.4% (HPLC area %).

### Example 5b

To a solution of the ester (1.0 g) in N,N-dimethylaniline (2.5 ml), taken in a 2 necked round bottom flask (RB) fitted with a water condenser and a nitrogen inlet, aqueous HCl (1 ml) was added and the reaction mixture was heated at 190° C. for 12 h. At the end of 12 h the reaction mixture was quenched with dilute HCl and the organics were extracted with diethyl ether and were analyzed by HPLC. Conversion was ˜100% and the purity of crude para-eugenol was 90.0% (HPLC area %).

### Example 5c

To a solution of the ester (1.0 g) in N,N-dimethylaniline (2.5 ml), taken in a 2 necked RB fitted with a water condenser and a nitrogen inlet, aniline hydrochloride (0.6 g) was added and the reaction mixture was heated at 190° C. for 3 h. At the end of 3 h the reaction mixture was quenched with dilute HCl and the organics were extracted with diethyl ether and were analyzed by HPLC. Conversion was 99% and the purity of crude para-eugenol was 81.0% (HPLC area %).

### Example 5d

To a solution of the ester (1.0 g) in N,N-dimethylformamide (2 ml), taken in a 2 necked RB fitted with a water condenser and a nitrogen inlet, aniline hydrochloride (0.6 g) was added and the reaction mixture was heated to reflux for 3 h. At the end of 3 h, the reaction mixture was quenched with water and the organics were extracted with diethyl ether and were analyzed by HPLC. Conversion was 98.6% and the purity of crude para-eugenol was 72% (HPLC area %).

### Example 6

### Synthesis of Eugenol End-Capped Polydimethylsiloxane

The following is one example of a procedure to prepare a eugenol end-capped polydimethylsiloxane. This procedure can be modified to use other amounts of components or variants of the components, as appreciated in the art.

Octamethylcyclotetrasiloxane (8.3 kg, 28.0 moles), tetramethyldisiloxane (330 g, 2.46 moles) and Filtrol 20 (86 g, 1% by weight) will be combined in a 12 L flask and heated to 45° C. for 2 hours. The temperature will be raised to 100° C. and the mixture rapidly agitated for 5 hours. The mixture will be allowed to cool then filtered through a plug of Celite filtering aid. To the crude product will be added a mixture of a product comprising para-eugenol (4.72 moles) and Karstedt's platinum catalyst (1.57 g, 10 ppm Pt) at a rate of 40 g/minute. Reaction completion will be monitored by the disappearance of the siloxane hydrogen in the Fourier-transform Infrared Spectroscopy (FTIR) spectrum. The reaction product can be stripped of volatiles using a falling thin film evaporator operating at 200 C and 1.5 torr. The material can be used without further purification.

### Example 7

### Preparation of Polysiloxane-Polycarbonate Copolymer Containing Synthetic Eugenol and 5.7 wt % Siloxane

The reaction process may be monitored for the presence and disappearance of reactants such as the siloxane bischloroformate and BPA. The presence of chloroformate is determined by placing approximately 1 ml of reactor organic phase onto paper tape that is impregnated with 4-(4-nitrobenzyl)pyridine. A yellow to orange color change indicates the presence of chloroformate groups.

The presence of unreacted BPA in reactor samples can be determined by diluting 2 ml of reactor sample organic phase in 5 ml dichloromethane. To the solution is added 5 ml dilute ammonium hydroxide and the mixture shaken vigorously for 30 seconds. To the mixture is added 10 ml of 1% aqueous potassium ferricyanide and the mixture shaken vigorously for 30 seconds. To the mixture is added 5 ml of 1% aqueous 4-aminoantipyrine and the mixture shaken vigorously for 30 seconds. A yellow color indicates acceptable low amount of residual BPA. An orange to red color indicates unacceptable high amount of residual BPA.

A solution program addition of the end cap and pre-phosgenation of a D45 siloxane monomer produced from synthetic eugenol in a tubular reactor was employed to obtain the polysiloxane polycarbonate copolymer.

A solution of p-cumylphenol (159 grams, 0.75 moles, 4.1 mole %) was prepared in 700 ml of dichloromethane. The p-cumylphenol (PCP) solution was placed in an addition pot connected to the reactor via a dosing pump.

A solution of eugenol capped D45 siloxane (312 g, 0.0082 mole, 5.7 wt % siloxane) was prepared in 900 ml of dichloromethane. The D45 siloxane solution was placed in an addition tank connected to the tubular reactor via a dosing pump. The tubular reactor (½ inch diameter×15 feet length, spiral upflow) was connected to the batch reactor.

To the formulation tank was added dichloromethane (13 L), DI water (8 L), bisphenol-A (4000 grams, 17.5 moles), triethylamine (40 grams, 0.39 moles) and sodium gluconate (10 grams, iron scavenger). The mixture was stirred for 5 minutes, then transferred to the 70 L batch reactor which was equipped with an overhead condenser, circulation loop, pH probe and various material addition nozzles. The formulation tank was rinsed with dichloromethane (5 L) which was transferred to the batch reactor. The reactor agitator was started and the circulation flow was set at 80 L/min. Phosgene vapor flow to the reactor was initiated (80 g/min flow rate) by the distributed control system (DCS) and an initial amount (220 grams, 2.2 moles) was added. The pH of the reaction was maintained at a target of 10.0 by DCS-controlled addition of 33% aqueous NaOH.

After addition of the initial amount of phosgene, the PCP solution was added to the reactor at 500 ml/min flow rate by DCS control while phosgene flow to the reactor continued. At the same time the feed to the tubular reactor was initiated with D45 siloxane solution flow (500 g/min) combining with phosgene (28 g/min, 0.28 mole/min) and 18% aqueous NaOH (316 g/min, 1.4 moles/min) in the plug flow reactor directly feeding into the batch reactor. The tubular reactor was flushed with dichloromethane (2 L). Phosgene addition to the batch reactor continued with pH control throughout the additions and until the total set point was reached (2200 grams, 22.2 moles). After completion of the phosgene addition, a sample of the reactor was obtained and verified to be free of un-reacted BPA and free of chloroformate. Weight average molecular weight (Mw) of a reaction sample was determined by GPC using a UV detector (Mw=23648, polydispersity index (PDI)=2.6). An additional charge of phosgene was added (200 grams, 2.0 mole) to the reactor. The reactor was purged with nitrogen then the batch was transferred to the centrifuge feed tank.

To the batch in the centrifuge feed tank was added dichloromethane (8 L), then the mixture was purified using a train of liquid-liquid centrifuges. Centrifuge one separated the brine phase. Centrifuge two removed the triethylamine catalyst by extracting the resin solution with aqueous hydrochloric acid (pH 1). Centrifuges three through eight removed residual ions by extracting the resin solution with DI water. A sample of the resin solution was tested and verified less than 5 ppm each of ionic chloride and residual triethylamine.

The resin solution was transferred to the precipitation feed tank. The resin was isolated as a white powder by steam precipitation followed by drying in a cone shaped vessel using heated nitrogen (210° 32 F). Powder yield: 3062 grams; Mw=23424; PDI=2.7. A hot pressed film of a resin powder sample was transparent and essentially haze free.

### Example 8

### Preparation of Polysiloxane-Polycarbonate Copolymer Containing Natural Eugenol and 5.7 wt % Siloxane

A control batch was produced using the exact sample reaction conditions with the exception that the D45 siloxane monomer was produced using natural source eugenol. The reactor product Mw was 23423, PDI=2.6. The Powder yield was 3266 grams. Mw=23410, PDI 2.6. A hot pressed film of a resin powder sample was transparent and essentially haze free.

### Example 9

### Preparation of Polysiloxane-Polycarbonate Copolymer Containing Synthetic Eugenol and 20.1 wt % Siloxane

A solution program addition of the end cap was employed to obtain the polysiloxane polycarbonate copolymer. A D45 siloxane monomer produced from synthetic eugenol was used in the synthesis.

A solution of p-cumylphenol (102 grams, 0.48 moles, 3.1 mole %) was prepared in 700 ml of dichloromethane. The p-cumylphenol (PCP) solution was placed in an addition pot connected to the reactor via a dosing pump.

To the formulation tank was added dichloromethane (13 L), DI water (10 L), bisphenol-A (3400 grams, 14.9 moles), synthetic eugenol D45 siloxane (1100 g, 0.29 mole, 20.1 weight % siloxane), triethylamine (36 grams, 0.35 moles) and sodium gluconate (10 grams, iron scavenger). The mixture was stirred for 5 minutes, then transferred to the 70 L batch reactor which was equipped with an overhead condenser, circulation loop, pH probe and various material addition nozzles. The formulation tank was rinsed with dichloromethane (5 L) which was transferred to the batch reactor. The reactor agitator was started and the circulation flow was set at 80 L/min. Phosgene vapor flow to the reactor was initiated (80 g/min flow rate) by the DCS and an initial amount (215 grams, 2.2 moles) was added. The pH of the reaction was maintained at a target of 10.0 by DCS-controlled addition of 33% aqueous NaOH.

After addition of the initial amount of phosgene, the PCP solution was added to the reactor at 500 ml/min flow rate by DCS control while phosgene flow to the reactor continued. Phosgene addition to the batch reactor continued with pH control throughout until the total set point was reached (2150 grams, 21.7 moles). After completion of the phosgene addition, a sample of the reactor was obtained and verified to be free of un-reacted BPA and free of chloroformate. Mw of a reaction sample was determined by GPC using a UV detector (Mw=30917, PDI=3.5). An additional charge of phosgene was added (200 grams, 2.0 mole) to the reactor. The reactor was purged with nitrogen then the batch was transferred to the centrifuge feed tank.

To the batch in the centrifuge feed tank was added dilution dichloromethane (8 L) then the mixture was purified using a train of liquid-liquid centrifuges. Centrifuge one separated the brine phase. Centrifuge two removed the triethylamine catalyst by extracting the resin solution with aqueous hydrochloric acid (pH 1). Centrifuges three through eight removed residual ions by extracting the resin solution with DI water. A sample of the resin solution was tested and verified less than 5 ppm each of ionic chloride and residual triethylamine.

The resin solution was transferred to the precipitation feed tank. The resin was isolated as a white powder by steam precipitation followed by drying in a cone shaped vessel using heated nitrogen (210° F.). Powder yield: 3198 grams. Mw=30361 PDI=2.8.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A process for synthesizing para-eugenol, comprising: a) hydrolyzing methyl 5-allyl-3-methoxysalicylate to form 5-allyl-3-methoxysalicylic acid; b) decarboxylating the 5-allyl-3-methoxysalicylic acid to form a product comprising para-eugenol.

Clause 2. The process of clause 1, wherein step a) comprises refluxing a mixture of methyl 5-allyl-3-methoxysalicylate in an aqueous base.

Clause 3. The process of clause 2, wherein the aqueous base comprises sodium hydroxide.

Clause 4. The process of any of clauses 2-3, wherein the aqueous base comprises a solution of about 10 wt % sodium hydroxide in water.

Clause 5. The process of any of clauses 2-4, wherein after the refluxing, step a) further comprises cooling the mixture and neutralizing the mixture with an acid.

Clause 6. The process of clause 5, wherein the acid is a mineral acid.

Clause 7. The process of any of clauses 5-6, wherein the acid comprises hydrochloric acid.

Clause 8. The process of any of clauses 5-7, wherein after cooling and neutralizing, step a) further comprises filtering and drying the 5-allyl-3-methoxysalicylic acid.

Clause 9. The process of any of clauses 1-8, wherein step b) comprises heating a mixture of 5-allyl-3-methoxysalicylic acid in a polar aprotic solvent, a base, or a combination thereof.

Clause 10. The process of clause 9, wherein the polar aprotic solvent is at least one of N,N-dimethylformamide and dimethylsulfoxide.

Clause 11. The process of any of clauses 9-10, wherein the base is at least one of substituted and unsubstituted aromatic amines.

Clause 12. The process of clause 9-11, wherein the base comprises N,N-dimethylaniline.

Clause 13. The process of clause 12, wherein the ratio by weight of the 5-allyl-3-methoxysalicylic acid to the N,N-dimethylaniline is from about 1:0.1 to about 1:3.

Clause 14. The process of any of clauses 9-13, wherein the mixture further comprises a metal salt.

Clause 15. The process of clause 14, wherein the metal salt is at least one alkali metal halide.

Clause 16. The process of clause 15, wherein the alkali metal halide comprises lithium chloride.

Clause 17. The process of clause 16, wherein the ratio by weight of the 5-allyl-3-methoxysalicylic acid to the lithium chloride is about 1:0.1 to about 1:1.

Clause 18. The process of any of clauses 16-17, wherein the ratio by weight of the 5-allyl-3-methoxysalicylic acid to the lithium chloride is about 1:0.1 to about 1:0.5.

Clause 19. The process of any of clauses 9-18, wherein the mixture is heated to reflux.

Clause 20. The process of any of clauses 9-19, wherein the mixture is heated for about 1 hour to about 12 hours.

Clause 21. The process of any of clauses 9-20, wherein step b) further comprises cooling the mixture and diluting the mixture with an organic solvent to provide a diluted organic mixture.

Clause 22. The process of clause 21, wherein the organic solvent is at least one of diethyl ether, chloroform, methylene chloride, ethyl acetate, benzene, toluene, pentane, hexane, cyclohexane, and 1,2-dichloroethane.

Clause 23. The process of any of clauses 21-22, wherein the organic solvent is 1,2-dichloroethane.

Clause 24. The process of any of clauses 21-23, further comprising adding a mineral acid to the diluted organic mixture and maintaining the temperature at less than about 25° C., and stirring the mixture.

Clause 25. The process of clause 24, wherein the mineral acid comprises aqueous hydrochloric acid.

Clause 26. The process of any of clauses 24-25, further comprising purifying the mixture by distillation, column chromatography, or a combination thereof.

Clause 27. The process of any of clauses 24-26, further comprising separating an organic layer from the mixture.

Clause 28. The process of clause 27, further comprising passing the organic layer through an acidic ion exchange resin to provide a neutralized organic layer.

Clause 29. The process of clause 28, further comprising purifying the product comprising para-eugenol from the neutralized organic layer by distillation.

Clause 30. The process of clause 29, wherein the distillation comprises high vacuum distillation.

Clause 31. The process of any of clauses 1-30, further comprising synthesizing a eugenol end-capped polydiorganosiloxane using the product comprising para-eugenol.

Clause 32. The process of clause 31, wherein the eugenol end-capped polydiorganosiloxane is a eugenol end-capped polydimethylsiloxane.

Clause 33. A process for synthesizing para-eugenol, comprising:
decarboxylating methyl 5-allyl-3-methoxysalicylate to form a product comprising para-eugenol.

Clause 34. The process of clause 33, wherein the decarboxylating step comprises providing a mixture of methyl 5-allyl-3-methoxysalicylate and a catalyst in a polar aprotic solvent or a base, and heating the mixture.

Clause 35. The process of clause 34, wherein the catalyst is at least one of alkali metal halides, aqueous mineral acids, and salts of aromatic amines.

Clause 36. The process of clause 35, wherein the alkali metal halide comprises lithium chloride.

Clause 37. The process of any of clauses 35-36, wherein the aqueous mineral acid is aqueous hydrochloric acid.

Clause 38. The process of any of clauses 35-37, wherein the salt of an aromatic amine is aniline hydrochloride.

Clause 39. The process of any of clauses 34-38, wherein the molar ratio of the methyl 5-allyl-3-methoxysalicylate and the catalyst is about 1:0.5 to about 1:1.

Clause 40. The process of any of clauses 34-39, wherein the polar aprotic solvent is at least one of N,N-dimethylformamide and dimethylsulfoxide.

Clause 41. The process of any of clauses 34-39, wherein the base is N,N-dimethylaniline.

Clause 42. The process of any of clauses 34-41, wherein the mixture is heated to reflux.

Clause 43. The process of any of clauses 34-42, wherein the mixture is heated for about 3 hours to about 12 hours.

Clause 44. The process of any of clauses 34-43, further comprising cooling the mixture and quenching the reaction with water or an aqueous acid to form a quenched reaction mixture.

Clause 45. The process of clause 44, wherein the aqueous acid is a mineral acid.

Clause 46. The process of clause 45, wherein the mineral acid is hydrochloric acid.

Clause 47. The process of any of clauses 44-46, further comprising extracting the para-eugenol from the quenched reaction mixture with an organic solvent.

Clause 48. The process of clause 47, wherein the organic solvent is at least one of diethyl ether, chloroform, methylene chloride, ethyl acetate, benzene, toluene, pentane, hexane, cyclohexane, and 1,2-dichloroethane.

Clause 49. The process of any of clauses 47-48, wherein the organic solvent is diethyl ether.

Clause 50. The process of any of clauses 47-49, further comprising evaporating the organic solvent to provide a crude product comprising para-eugenol.

Clause 51. The process of clause 50, further comprising synthesizing a eugenol end-capped polydiorganosiloxane from the crude product.

Clause 52. The process of clause 51, wherein the eugenol end-capped polydiorganosiloxane is a eugenol end-capped polydimethylsiloxane.

Clause 53. The process of clause 50, further comprising purifying the crude product via distillation to provide a purified product comprising para-eugenol.

Clause 54. The process of clause 53, wherein the distillation comprises high vacuum distillation.

Clause 55. The process of any of clauses 53-54, further comprising synthesizing a eugenol end-capped polydiorganosiloxane from the purified product.

Clause 56. The process of clause 55, wherein the eugenol end-capped polydiorganosiloxane is a eugenol end-capped polydimethylsiloxane.

Clause 57. A process for synthesizing para-eugenol, comprising: a) providing a first mixture comprising a catalytic amount of a Group IB metal salt and a nitrogen-containing compound; b) cooling the first mixture to about 0° C. to about 15° C.; c) adding about 0.8 to about 1 equivalent of an alkali metal halide to the first mixture to form a second mixture; d) adding an aqueous solution of about 0.7 to about 1 equivalent of a base to the second mixture to form a third mixture; e) adding about 1 equivalent of guaiacol to the third mixture dropwise to form a fourth mixture; f) allowing the fourth mixture to warm to a temperature of about 15° C. to about 25° C.; g) adding about 1.0 to about 1.5 equivalents of an allyl halide to the fourth mixture, maintaining a temperature of less than about 30° C., to form a fifth mixture comprising para-eugenol.

Clause 58. The process of clause 57, wherein the Group IB metal salt is a copper salt.

Clause 59. The process of clause 58, wherein the copper salt is copper(I) chloride.

Clause 60. The process of any of clauses 57-59, wherein the nitrogen-containing compound is at least one of ammonia and salts thereof, an amine, an amino acid, an amide, a hydrazine, and a basic cross-linked polystyrene resin.

Clause 61. The process of clause 60, wherein the nitrogen-containing compound is aqueous ammonia.

Clause 62. The process of any of clauses 57-61, wherein step b) comprises cooling the first mixture to about 10° C.

Clause 63. The process of any of clauses 57-62, wherein the alkali metal halide is sodium iodide.

Clause 64. The process of any of clauses 57-63, wherein step c) comprises adding about 0.9 equivalent of sodium iodide to the first mixture.

Clause 65. The process of any of clauses 57-64, wherein step c) further comprises maintaining a temperature of less than about 15° C. during the adding of the alkali metal halide.

Clause 66. The process of any of clauses 57-65, wherein the base is at least one of hydroxides, bicarbonates, carbonates and acetates of alkali and alkaline earth metals.

Clause 67. The process of any of clauses 57-66, wherein the base is sodium hydroxide.

Clause 68. The process of any of clauses 57-67, wherein the allyl halide is allyl chloride.

Clause 69. The process of any of clauses 57-68, wherein step f) comprises allowing the fourth mixture to warm to a temperature of about 20° C. to about 22° C.

Clause 70. The process of any of clauses 57-69, wherein step g) comprises maintaining a temperature of between about 20° C. and about 25° C.

Clause 71. The process of any of clauses 57-70, wherein step g) further comprises stirring the fifth mixture for about 5 minutes to about 15 minutes.

Clause 72. The process of any of clauses 57-71, further comprising neutralizing the fifth mixture by adding a mineral acid dropwise and maintaining the temperature at about 25° C., to form a sixth mixture.

Clause 73. The process of clause 72, wherein the mineral acid is an aqueous solution of hydrochloric acid.

Clause 74. The process of any of clauses 72-73, further comprising extracting the sixth mixture with an organic solvent to provide an organic extract.

Clause 75. The process of clause 74, wherein the organic solvent is at least one of diethyl ether, chloroform, methylene chloride, ethyl acetate, benzene, toluene, pentane, hexane, cyclohexane, and 1,2-dichloroethane.

Clause 76. The process of any of clauses 74-75, wherein the organic solvent is diethyl ether.

Clause 77. The process of any of clauses 74-76, further comprising drying the organic extract and removing the organic solvent to provide a crude product.

Clause 78. The process of clause 77, further comprising dissolving the crude product in a basic aqueous solution and extracting the basic aqueous solution with an organic solvent to provide an extracted aqueous solution.

Clause 79. The process of clause 78, further comprising neutralizing the extracted aqueous solution with an acid to provide a neutralized aqueous solution, and extracting the neutralized organic solution with an organic solvent to provide a second organic extract.

Clause 80. The process of clause 79, further comprising drying the second organic extract and removing the organic solvent to provide a product mixture.

Clause 81. The process of clause 80, further comprising purifying the product mixture via distillation to provide a purified product mixture.

Clause 82. The process of clause 81, wherein the distillation comprises high vacuum distillation.

Clause 83. The process of any of clauses 80-82, wherein the product mixture or the purified product mixture comprises about 70-95% para-eugenol and about 5-30% ortho-eugenol.

Clause 84. The process of clause 83, wherein the product mixture or the purified product mixture comprises about 75-90% para-eugenol and about 10-25% ortho-eugenol.

Clause 85. The process of any of clauses 83-84, wherein the product mixture or the purified product mixture comprises about 80-85% para-eugenol and about 15-20% ortho-eugenol.

Clause 86. The process of any of clauses 80-85, further comprising synthesizing a eugenol end-capped polydiorganosiloxane from the product mixture or the purified product mixture.

Clause 87. The process of clause 86, wherein the eugenol end-capped polydiorganosiloxane is a eugenol end-capped polydimethylsiloxane.

Clause 88. The process of clause 87, further comprising synthesizing a eugenol end-capped polydimethylsiloxane bischloroformate from the eugenol end-capped polydimethylsiloxane.

Clause 89. The process of clause 88, further comprising synthesizing a polysiloxane-polycarbonate copolymer, wherein the method for synthesizing the polysiloxane-polycarbonate copolymer comprises: (a) contacting a dihydroxyaromatic compound with a continuous supply of phosgene under interfacial reaction conditions, in aqueous-organic solvent at a pH of about 9-12, in the presence of at least one tertiary amine, to form a mixture; (b) combining a monohydroxyaromatic compound with the mixture to produce an oligomeric aromatic polycarbonate, with additional introduction of phosgene; (c) combining a mixture of the eugenol end-capped polydimethylsiloxane bischloroformate in aqueous-organic solvent with the oligomeric aromatic polycarbonate, with additional introduction of phosgene, to form a reaction mixture; and (d) adding the remainder of the phosgene to the reaction mixture, while stirring, to afford a polysiloxane-polycarbonate copolymer of a desired molecular weight.

Clause 90. The process of clause 89, wherein the dihydroxyaromatic compound is bisphenol A (BPA).

Clause 91. The process of any one of clause 89 or clause 90, wherein the monohydroxyaromatic compound is selected from the group consisting of phenol and para-cumylphenol (PCP), or a combination thereof.

Clause 92. The process of any one of clauses 89-91, wherein the monohydroxyaromatic compound is para-cumylphenol (PCP).

Clause 93. The process of any one of clauses 89-92, wherein the oligomeric aromatic polycarbonate is a BPA polycarbonate.

Clause 94. The process of any one of clauses 89-93, wherein the oligomeric BPA polycarbonate is a PCP end-capped BPA polycarbonate.

Clause 95. The process of any one of clauses 89-94, wherein the monohydroxyaromatic compound of step (b) is added to the mixture of step (a).

Clause 96. The process of any one of clauses 89-95, wherein the eugenol end-capped polydimethylsiloxane bischloroformate is added to the oligomeric aromatic polycarbonate.

Clause 97. The process of any one of clauses 89-96, wherein the polysiloxane-polycarbonate copolymer is a siloxane block co-polycarbonate comprising, by weight, about 2% siloxane to about 30% siloxane.

Clause 98. The process of any one of clauses 89-97, wherein the polysiloxane-polycarbonate copolymer is a siloxane block co-polycarbonate comprising, by weight, about 3% siloxane to about 20% siloxane.

Clause 99. The process of any one of clauses 89-98, wherein the polysiloxane-polycarbonate copolymer is a siloxane block co-polycarbonate comprising, by weight, about 3% siloxane to about 8% siloxane.

Clause 100. The process of any one of clauses 89-99, wherein the polysiloxane-polycarbonate copolymer is a siloxane block co-polycarbonate comprising, by weight, about 6% siloxane.

Clause 101. The process of any one of clauses 89-100, wherein the polysiloxane-polycarbonate copolymer is a PCP end-capped BPA polycarbonate-polydimethylsiloxane copolymer having an average polydimethylsiloxane block length of 45 units, and having a weight average molecular weight of about 17,000 g/mol to about 50,000 g/mol, as determined by gel permeation chromatography (GPC) using BPA polycarbonate standards.

Clause 102. The process of any one of clauses 89-101, wherein the polysiloxane-polycarbonate copolymer is a PCP end-capped BPA polycarbonate-polydimethylsiloxane copolymer having an average polydimethylsiloxane block length of 45 units, and having a weight average molecular weight of about 20,000 g/mol to about 40,000 g/mol, as determined by gel permeation chromatography (GPC) using BPA polycarbonate standards.

Clause 103. The process of any one of clauses 89-102, wherein the polysiloxane-polycarbonate copolymer is a PCP end-capped BPA polycarbonate-polydimethylsiloxane copolymer having an average polydimethylsiloxane block length of 45 units, and having a weight average molecular weight of about 22,000 g/mol to about 32,000 g/mol, as determined by gel permeation chromatography (GPC) using BPA polycarbonate standards.

Clause 104. The process of any one of clauses 89-98 or 101-103, wherein the polysiloxane-polycarbonate copolymer is a PCP end-capped BPA polycarbonate-polydimethylsiloxane copolymer comprising about 20 wt % siloxane, having an average polydimethylsiloxane block length of 45 units, and having a weight average molecular weight of about 30,000 g/mol to about 32,000 g/mol, as determined by gel permeation chromatography (GPC) using BPA polycarbonate standards.

Clause 105. The process of any one of clauses 89-103, wherein the polysiloxane-polycarbonate copolymer is a PCP end-capped BPA polycarbonate-polydimethylsiloxane copolymer comprising about 6 wt % siloxane, having an average polydimethylsiloxane block length of 45 units, and having a weight average molecular weight of about 22,000 g/mol to about 24,000 g/mol, as determined by gel permeation chromatography (GPC) using BPA polycarbonate standards.

Clause 106. The process of clause 87, further comprising synthesizing a polysiloxane-polycarbonate copolymer, wherein the method for synthesizing the polysiloxane-polycarbonate copolymer comprises: (a) combining a dihydroxyaromatic compound, the eugenol end-capped polydiorganosiloxane and an initial amount of phosgene under interfacial reaction conditions, in aqueous-organic solvent at a pH of about 9-12, in the presence of at least one tertiary amine, to form a mixture; (b) combining a monohydroxyaromatic compound and the mixture, with additional introduction of phosgene to form a reaction mixture; and (c) adding the remainder of the phosgene to the reaction mixture to afford a polysiloxane-polycarbonate copolymer of a desired molecular weight.

Clause 107. The process of clause 106, wherein the dihydroxyaromatic compound is bisphenol A (BPA).

Clause 108. The process of any one of clause 106 or clause 107, wherein the monohydroxyaromatic compound is selected from the group consisting of phenol and para-cumylphenol (PCP).

Clause 109. The process of any one of clauses 106-108, wherein the monohydroxyaromatic compound is para-cumylphenol (PCP).

Clause 111. The process of any one of clauses 106-110, wherein the polysiloxane-polycarbonate copolymer is a siloxane block co-polycarbonate comprising, by weight, about 3% siloxane to about 20% siloxane.

Clause 112. The process of any one of clauses 106-111, wherein the polysiloxane-polycarbonate copolymer is a siloxane block co-polycarbonate comprising, by weight, about 12% siloxane to about 25% siloxane.

Clause 113. The process of any one of clauses 106-112, wherein the polysiloxane-polycarbonate copolymer is a siloxane block co-polycarbonate comprising, by weight, about 20% siloxane.

Clause 114. The process of any one of clauses 106-113, wherein the polysiloxane-polycarbonate copolymer is a PCP end-capped BPA polycarbonate-polydimethylsiloxane copolymer having an average polydimethylsiloxane block length of 45 units, and having a weight average molecular weight of about 17,000 g/mol to about 50,000 g/mol, as determined by gel permeation chromatography (GPC) using BPA polycarbonate standards.

Clause 115. The process of any one of clauses 106-114, wherein the polysiloxane-polycarbonate copolymer is a PCP end-capped BPA polycarbonate-polydimethylsiloxane copolymer having an average polydimethylsiloxane block length of 45 units, and having a weight average molecular weight of about 20,000 g/mol to about 40,000 g/mol, as determined by gel permeation chromatography (GPC) using BPA polycarbonate standards.

Clause 116. The process of any one of clauses 106-115, wherein the polysiloxane-polycarbonate copolymer is a PCP end-capped BPA polycarbonate-polydimethylsiloxane copolymer having an average polydimethylsiloxane block length of 45 units, and having a weight average molecular weight of about 22,000 g/mol to about 32,000 g/mol, as determined by gel permeation chromatography (GPC) using BPA polycarbonate standards.

Clause 117. The process of any one of clauses 106-116, wherein the polysiloxane-polycarbonate copolymer is a PCP end-capped BPA polycarbonate-polydimethylsiloxane copolymer comprising about 20 wt % siloxane, having an average polydimethylsiloxane block length of 45 units, and having a weight average molecular weight of about 30,000 g/mol to about 32,000 g/mol, as determined by gel permeation chromatography (GPC) using BPA polycarbonate standards.

Clause 118. The process of any one of clauses 106-111 or 114-116, wherein the polysiloxane-polycarbonate copolymer is a PCP end-capped BPA polycarbonate-polydimethylsiloxane copolymer comprising about 6 wt % siloxane, having an average polydimethylsiloxane block length of 45 units, and having a weight average molecular weight of about 22,000 g/mol to about 24,000 g/mol, as determined by gel permeation chromatography (GPC) using BPA polycarbonate standards.

1. A process for synthesizing para-eugenol, comprising:
a) hydrolyzing methyl 5-allyl-3-methoxysalicylate to form 5-allyl-3-methoxysalicylic acid;
b) decarboxylating the 5-allyl-3-methoxysalicylic acid to form a product comprising para-eugenol.

2. The process of claim 1, wherein step a) comprises refluxing a mixture of methyl 5-allyl-3-methoxysalicylate in an aqueous base.

3. The process of claim 2, wherein after the refluxing, step a) further comprises cooling the mixture and neutralizing the mixture with an acid.

4. The process of claim 3, wherein after cooling and neutralizing, step a) further comprises filtering and drying the 5-allyl-3-methoxysalicylic acid.

5. The process of claim 1, wherein step b) comprises heating a mixture of 5-allyl-3-methoxysalicylic acid in a polar aprotic solvent, a base, or a combination thereof.

6. The process of claim 5, wherein step b) further comprises cooling the mixture and diluting the mixture with an organic solvent to provide a diluted organic mixture.

7. The process of claim 6, further comprising adding a mineral acid to the diluted organic mixture and maintaining the temperature at less than about 25° C., and stirring the mixture.

8. The process of claim 7, wherein the mineral acid comprises aqueous hydrochloric acid.

9. A process for synthesizing para-eugenol, comprising:
decarboxylating methyl 5-allyl-3-methoxysalicylate to form a product comprising para-eugenol.

10. The process of claim 9, wherein the decarboxylating step comprises providing a mixture of methyl 5-allyl-3-methoxysalicylate and a catalyst in a polar aprotic solvent or a base, and heating the mixture.

11. A process for synthesizing para-eugenol, comprising:
a) providing a first mixture comprising a catalytic amount of a Group IB metal salt and a nitrogen-containing compound;
b) cooling the first mixture to about 0° C. to about 15° C.;
c) adding about 0.8 to about 1 equivalent of an alkali metal halide to the first mixture to form a second mixture;
d) adding an aqueous solution of about 0.7 to about 1 equivalent of a base to the second mixture to form a third mixture;
e) adding about 1 equivalent of guaiacol to the third mixture dropwise to form a fourth mixture;
f) allowing the fourth mixture to warm to a temperature of about 15° C. to about 25° C.;
g) adding about 1.0 to about 1.5 equivalents of an allyl halide to the fourth mixture, maintaining a temperature of less than about 30° C., to form a fifth mixture comprising para-eugenol.

12. The process of claim 11, wherein step b) comprises cooling the first mixture to about 10° C.

13. The process of claim 11, wherein step c) comprises adding about 0.9 equivalent of sodium iodide to the first mixture.

14. The process of claim 11, wherein step c) further comprises maintaining a temperature of less than about 15° C. during the adding of the alkali metal halide.

15. The process of claim 11, wherein step f) comprises allowing the fourth mixture to warm to a temperature of about 20° C. to about 22° C.

16. The process of claim 11, wherein step g) comprises maintaining a temperature of between about 20° C. and about 25° C.

17. The process of claim 11, wherein step g) further comprises stirring the fifth mixture for about 5 minutes to about 15 minutes.

18. The process of claim 11, further comprising neutralizing the fifth mixture by adding a mineral acid dropwise and maintaining the temperature at about 25° C., to form a sixth mixture.

19. The process of claim 18, further comprising extracting the sixth mixture with an organic solvent to provide an organic extract.

20. The process of claim 19, further comprising drying the organic extract and removing the organic solvent to provide a crude product.
